(19) United States
(12) Patent Application Publication
(10) Pub. No.: US 2021/0299257 A1
(43) Pub. Date: Sep. 30, 2021

(54) Methods, Systems and Apparatus for Reducing Pathogen Loads in Circulating Body Fluids

(71) Applicant: Mi2 Holdings LLC, Houston, TX (US)

(72) Inventors: Andrew Hopkins, Houston, TX (US); Thomas Hopkins, Houston, TX (US)

(73) Assignee: Mi2 Holdings LLC, Houston, TX (US)

(21) Appl. No.: 17/207,226

(22) Filed: Mar. 19, 2021

Related U.S. Application Data
(60) Provisional application No. 62/994,693, filed on Mar. 25, 2020.
(60) Provisional application No. 63/008,655, filed on Apr. 10, 2020.

(51) Int. Cl. A61K 41/00; A61M 1/36; A61K 9/51; A61K 38/48; A61N 5/06; A61K 38/08; A61K 38/10

(52) U.S. Cl. CPC A61K 41/0057; A61M 1/3683; A61K 9/5146; A61K 38/4813; A61N 2005/067; A61N 5/0624; A61K 38/08; A61K 38/10; A61N 5/062

(57) ABSTRACT

A nanocomposition for use in treating a pathogen condition using phthalocyanine dye, such as IR700. A nanocomposition having IR700, an 8PEG nanoparticle and a pathogen targeting peptide. Administering a product comprising IR700 to a patient, whereby the IR700 is delivered to pathogen tissue, and found in only pathogen tissue; and administering light to activate the IR700, thereby producing an ROS.

### CROSS REFERENCE TO RELATED APPLICATIONS

This application claims furthers the disclosure and teachings of provisional application 62/994,693 filed Mar. 25, 2020, the entire disclosure of which is incorporated herein by reference.

### BACKGROUND

The present inventions relate generally to targeted photodynamic therapies, targeted photosensitizers, including targeted nanoconstructs and uses of these therapies and materials in dynamic therapies for treating, managing, reducing and eliminating pathogens in body fluids and from animals including humans. In particular, in an embodiment, the present inventions related to the removal of pathogens from circulating blood in animals, including humans.

The terms “nanocomposition”, “nanoparticle”, “nanomaterial”, “nanoparticle”, nanoproduct”, “nanoplatform”, “nano construct”, “nanocomposite”, “nano”, and similar such terms, unless specified otherwise, are to be given their broadest possible meaning, and include particles, materials and compositions having a volumetric shape that has at least one dimension from about 1 nanometer (nm) to about 100 nm. Preferably, in embodiments, these volumetric shapes have their largest cross section from about 1 nm to about 100 nm.

The terms “nanocomposition”, “nanoconstructs”, “nanoplatform”, “nanocomposite”, and “nano construct” and similar such terms, unless specified otherwise, are to be given their broadest possible meaning, and include a particle having a backbone material, e.g., a cage, support or matrix material, and one or more additives, e.g., agents, moieties, compositions, biologics, and molecules, that are associated with the backbone. Generally, the backbone material can be a nanoparticle. Generally, the additive is an active material having targeting, therapeutic, imaging, diagnostic, theranostic or other capabilities, and combinations and variations of these. In embodiments, the backbone material can be an active material, having targeting, therapeutic, imaging, diagnostic, theranostic or other capabilities, and combinations and variations of these. In embodiments both the additive and the backbone material are active materials. One, two, three or more different types of backbone materials, additives and combination and variations of these are contemplated.

The term “theranostic”, unless specified otherwise, is to be given its broadest possible meaning, and includes a particle, agent, composition, or material that has multiple capabilities and functions, including both imaging and therapeutic capabilities, both diagnostic and therapeutic capabilities, and combinations and variations of these and other features such as targeting.

The terms “imaging”, “imaging agent”, “imaging apparatus” and similar such terms, unless specified otherwise, should be given their broadest possible meaning, and would include apparatus, agents and materials that enhance, provide or enable the ability to detect, analyze and visualize the size, shape, position, composition, and combinations and variations of these as well as other features, of a structure, and in particular structures in animals, mammals and humans. Imaging agents would include contrast agents, dies, and similar types of materials. Examples of imaging apparatus and methodologies include x-ray; magnetic resonance; computer axial tomography scan (CAT scan); proton emission tomography scan (PET scan); ultrasound; florescence; and photo acoustic.

The term, “diagnostic”, unless specified otherwise, is to be given its broadest possible meaning, and would include identifying, determining, defining and combinations and variations of these, conditions, diseases and both, including conditions and diseases of animals, mammals and humans.

The term “therapeutic” and “therapy” and similar such terms, unless specified otherwise, are to be given their broadest possible meaning and would include addressing, treating, managing, mitigating, curing, preventing, and combinations and variations of these, conditions and diseases, including conditions and disease of animals, mammals and humans.

The terms “photodynamic therapy”, “PDT” and similar such terms, unless expressly stated otherwise, are to be given their broadest possible meaning and would include a method for ablating, (e.g., killing, destroying, rendering inert), biological tissue by photo-oxidation utilizing photosensitizer (“PS”) molecules. When the photosensitizer is exposed to a specific wavelength or wavelengths of light, it produces a form of oxygen from adjacent (e.g., in situ, local, intercellular, intracellular) oxygen sources, that kills nearby cells, e.g., reactive oxygen species (“ROS”), which includes any form of oxygen that are cyto-toxic to cells. It being understood that while light across all wavelengths, e.g., UV to visible to IR, is generally used as the activator of the PS, PS typically have a wavelength, or wavelengths where their absorption is highest.

The terms “activation dynamic therapy”, “dynamic therapy”, “dynamic therapy agent” and similar such terms, unless expressly stated otherwise, should be given their broadest possible meaning and would include PDT and PS, as well as agents that are triggered to product active oxygen, such as a reactive oxygen species (“ROS”) or other active therapeutic materials, when exposed to energy sources including energy sources other than light, as activators. These would include materials or agents that are activated by energy sources such as radio waves, other electromagnet radiation, magnetism, and sonic (e.g., Sonodynamic therapy or SDT).

The terms “photosensitizer” and “PS” and “photoactive agent” and similar such terms, unless expressly stated otherwise, should be given their broadest possible meaning and would include any dye, molecule or modality that when exposed to light produces, or causes the production of ROS, or other active agents that are cyto-toxic to cells, kill tissue, ablates tissue, destroys tissue or renders a pathogen inert.

The terms “targeting agent” and “TA” and similar such terms, unless expressly stated otherwise, should be given their broadest possible meaning and would include any molecule, material or modality that is targeted to, or specific for, or capable of binding to or with, a predetermined cell type, receptor, or pathogen. TA would include, for example, a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations and variations of these.

As used herein, unless expressly stated otherwise the term “pathogen” should be given its broadest possible means in would include any organism that can cause a disease or condition in animals (including humans, pets and livestock) or plants. Pathogens would include, for example, viruses, bacteria, fungi, molds, and parasites. Pathogens would include, for example, among others influenza viruses, corona viruses, COVID-19, SARS-CoV-2, Ebola, HIV, SARS, H1N1 and MRSA.

The term “antibody” as used herein, unless specified otherwise, should be given its broadest possible meaning, and would include a polypeptide ligand comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (VH) region and the variable light (V_{L}) region. Together, the VH region and the V_{L} region are responsible for binding the antigen recognized by the antibody. Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). The term antibody would include monoclonal antibodies, chimeric antibodies, and humanized immunoglobulin, to name a few.

As used herein, unless stated otherwise, room temperature is 25° C. and, standard ambient temperature and pressure is 25° C. and 1 atmosphere. Unless expressly stated otherwise all tests, test results, physical properties, and values that are temperature dependent, pressure dependent, or both, are provided at standard ambient temperature and pressure, this would include viscosities.

Generally, the term “about” and the symbol “˜” as used herein unless stated otherwise is meant to encompass a variance or range of ±10%, the experimental or instrument error associated with obtaining the stated value, and preferably the larger of these.

As used herein, unless specified otherwise, the recitation of ranges of values, a range, from about “x” to about “y”, and similar such terms and quantifications, serve as merely shorthand methods of referring individually to separate values within the range. Thus, they include each item, feature, value, amount or quantity falling within that range. As used herein, unless specified otherwise, each and all individual points within a range are incorporated into this specification, and are a part of this specification, as if they were individually recited herein.

As used herein, unless expressly stated otherwise terms such as “at least”, “greater than”, also mean “not less than”, i.e., such terms exclude lower values unless expressly stated otherwise.

This Background of the Invention section is intended to introduce various aspects of the art, which may be associated with embodiments of the present inventions. Thus, the forgoing discussion in this section provides a framework for better understanding the present inventions, and is not to be viewed as an admission of prior art.

### SUMMARY

There has been a long-standing and unfulfilled need for new and innovative drugs, medical products and medical systems and methods to address-pathogenic conditions in animals, mammals, and humans. This long-standing and unfulfilled need is present, and of critical concern for pandemics, conditions where no therapy is available, and other conditions where high mortality rates may exist. This long standing and critical need was present in the 1918 Spanish flu pandemic, with the AIDS/HIV epidemic, with Ebola, is present with the 2020 COVID-19 pandemic.

The present inventions, among other things, solve these needs by providing the compositions, materials, articles of manufacture, devices, methods and processes taught, disclosed and claimed herein.

### BRIEF DESCRIPTION OF THE DRAWINGS

FIG. 1 is a schematic formulaic representation of embodiments of targeted delivery nanocompositions, systems and products, in accordance with the present inventions.

FIG. 2 is a schematic formulaic representation of embodiments of various NP, TA and PS parings and combinations in accordance with the present inventions.

FIG. 3 is a formulaic representation of embodiments of linkers and functional group conversions in accordance with the present inventions.

FIG. 4 is a schematic formulaic representation of a nanocomposition in accordance with the present inventions.

FIG. 5A is a flow diagram of an embodiment of a process for making an embodiment of a nanocomposition in accordance with the present inventions.

FIG. 5B is a flow diagram of an embodiment of a process for making an embodiment of a nanocomposition in accordance with the present inventions.

FIG. 6A is a flow diagram of an embodiment of a process for making an embodiment of a PS for use in making a nanocomposition in accordance with the present inventions.

FIG. 6B is a flow diagram of an embodiment of a process for making an embodiment of a nanocomposition in accordance with the present inventions.

FIG. 7 is a schematic illustrating an embodiment of a PDT therapy in accordance with the present inventions.

FIG. 8 is a schematic view of a system and method of PDT therapies in accordance with the present inventions.

FIG. 9 is a schematic view of a system and method of PDT therapies in accordance with the present inventions.

FIG. 10 is a prospective view of an illumination device in accordance with the present inventions.

FIG. 11 is a prospective view of an illumination device in accordance with the present inventions.

FIG. 12 is an illustration of a virus to be treated in accordance with the present inventions.

### DETAILED DESCRIPTION

The present disclosure relates to the use of a targeted photosensitizer and targeted photodynamic therapies for treating, therapies, mitigating, managing, and eliminating pathogens, reducing viral loads, in bodily fluids, in vivo, ex-vivo and while circulating or standing.

Embodiments of the present inventions relate generally to targeted photodynamic therapies, targeted photosensitizers, including targeted nanoconstructs and uses of these therapies and materials in dynamic therapies for treating, managing, reducing, and eliminating pathogens in body fluids and from animals including humans. In an embodiment, the present inventions related to the removal of pathogens from circulating blood in animals, including humans.

Viruses have been estimated to be the most abundant and diverse biological systems on earth. Viruses may range in size from about 20 nm-about 300 nm. Viruses depend on living cells for their reproduction and are classified according to their genome and method of reproduction (Baltimore classification). They consist of a DNA or RNA (single or double stranded) core an outer protein cover and in some virus classes, lipids.

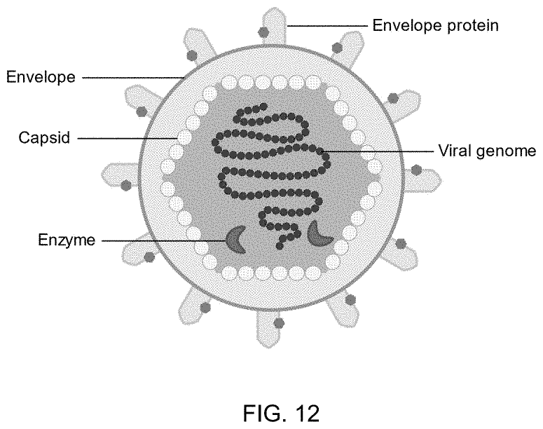

Turning to FIG. 12 there is shown a simplified diagram of the structure of a virus. When a virus infects a cell, the virus forces it to make thousands more viruses. It does this by making the cell copy the virus's DNA or RNA, making viral proteins, which all assemble to form new virus particles. When a virus infects a cell, the virus forces it to make thousands more viruses. It does this by making the cell copy the virus's DNA or RNA, making viral proteins, which all assemble to form new virus particles. Viruses can have a variety of effects on their cell “hosts”, although most infections usually result in cell death. Some viruses cause no apparent change and are said to be “latent”—this can cause a persistent infection with the virus lying dormant for many months or years (e.g., Herpes Viruses). One way of tracking the presence, persistence and progression of a viral infection is to measure the “viral load”—this is a test that is performed to measure the number of viral particles in a volume of fluid—usually serum. The lower the concertation per unit volume of fluid, i.e., titer, the better—as it is circulating viruses that cause the infection—they must circulate to find the right cell (and this receptor) to bind with. One effective way to manage a viral infection would be reduce this titer to the lowest possible number—while other symptoms caused by the infection of the host cell are addressed, and to do this in a simple minimally invasive (and potentially as an out-patient) therapy.

Thus, in an embodiment the present disclosure provides a PDT (photodynamic) composition as well as methods and systems to the PDT composition in targeting and killing of particles in the bloodstream. These therapies reduce the viral load to a point where symptoms or harm from the virus are reduced, preferably where symptoms and harm from the virus are eliminated, and where the virus is eliminated from the patient. In embodiments the viral load can be reduced by 50%, 60%, 70%, 80% 90% and 100% in a single treatment. The treatments can be repeated; thus, the PDT composition can be administered one, two, three or more times, the blood can be illuminated one, two, three or more times, and combinations and variations of these multi-step treatments.

Regardless of the method of transmission to the host the virus will circulate in the body until it is able to locate and bind to a cell expressing it receptor of choice when it will bind to that receptor and “infect” the cell. It is believed that the smaller the number of viral cells present the lower the risk of devastating infection. Thus, reducing the number of virus particles present in the host should reduce the severity of the infection.

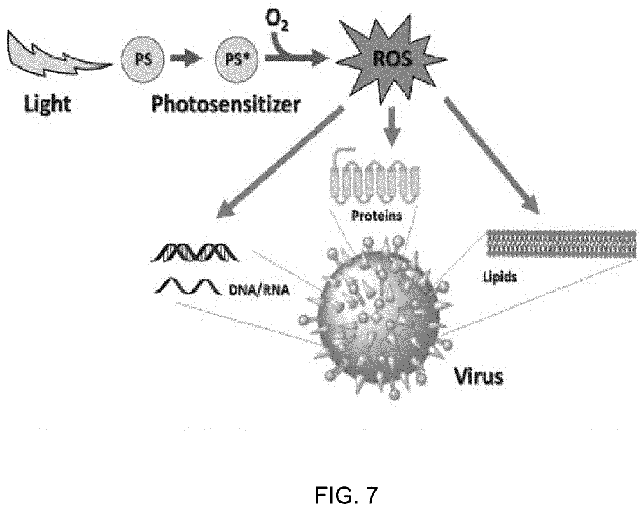

Turning to FIG. 7 there is shown a schematic of a targeted Photodynamic therapy PDT method to kill a virus. The PDT method is unspecific as it is not targeted to any particular virus classes. The PDT method may utilize ROS (reactive oxygen species) generation that can attack any part of the Viral particle thereby removing any possibility of evolving drug resistance. The method of viral particle destruction using PDT will release “fragments” (antigens) that can stimulate an immune response, by the body, to the parent virus creating the ability to combat the virus at the site of infection as well. Thus, the present therapies have a two-phase treatment effect. First the targeted PDT directly reduces the virus load. Second, the targeted PDT therapy stimulates or enhances an immune response allowing the body to continue to fight the virus after, and long after, the targeted PDT therapy has ended. Thus, this second treatment effect also provides the ability for the body to develop an immunity to the virus. Embodiments of the targeted PDT therapies, compositions, and systems of the present invention address three objectives, among others, for the treatment of virus in the blood. First, these embodiments target only the viral particle so that only the virus is destroyed. Second, these embodiments deliver the correct light dosage preferably in a simple, minimally invasive way. Third, these embodiments provide systems, devices, methods, and composition that achieve the first two objectives.

Embodiments of the present disclosure may further relate to devices to deliver light to activate photosensitizer to produce ROS in circulating blood, ex vivo and in vivo; as well as therapies and treatments using these devices. The blood may be removed, and a dose of the PDT composition may be administered to the patient, or the PDT composition may be added to the blood after removal. The blood may be held in an illumination device, illuminated to produce ROS, and then returned to patient. In an embodiment, the blood, after a dose of the targeted-photosensitizer composition has been administered to the patient, is removed on a continuous basis, e.g., circulating blood, moved through an illumination device, where the photosensitizer is activated by exposing the blood to the light and thereby producing ROS in the direct vicinity of the pathogen, e.g., the virus.

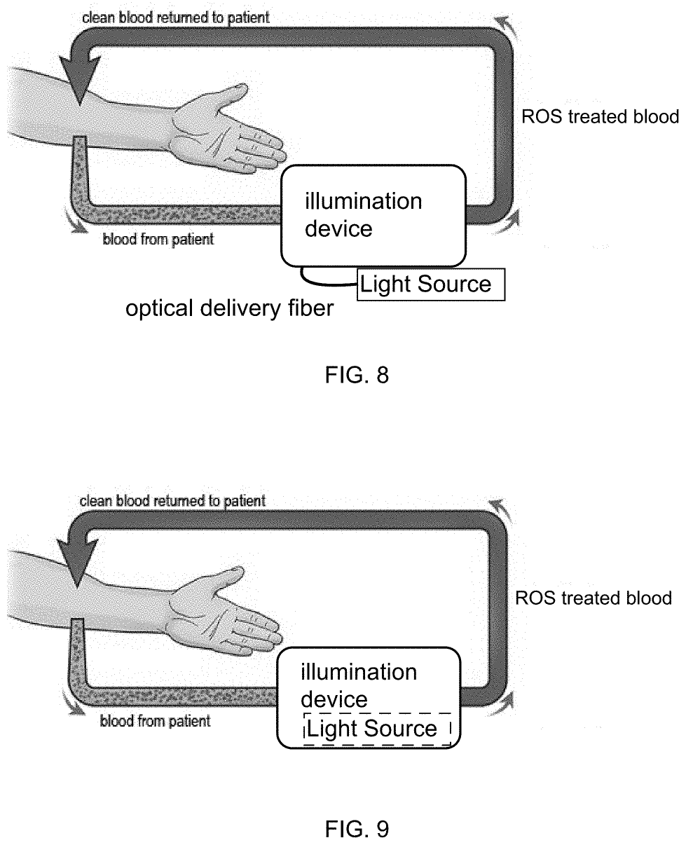

Turning to FIG. 8 there is a schematic of a therapy and system using the present targeted PDT composition. The patient has been provided with a therapeutic dose of a targeted PDT composition, that is targeted for a specific pathogen. After a sufficient period of time for the dose to distribute through the blood the blood is removed from the patient and flows through tubing to an illumination device, where the blood is illuminated. In this manner the PDT composition in the blood is activated delivering ROS to the virus.

FIG. 9 shows an embodiment of a schematic of a therapy and system illumination device where the light source is integral with the device.

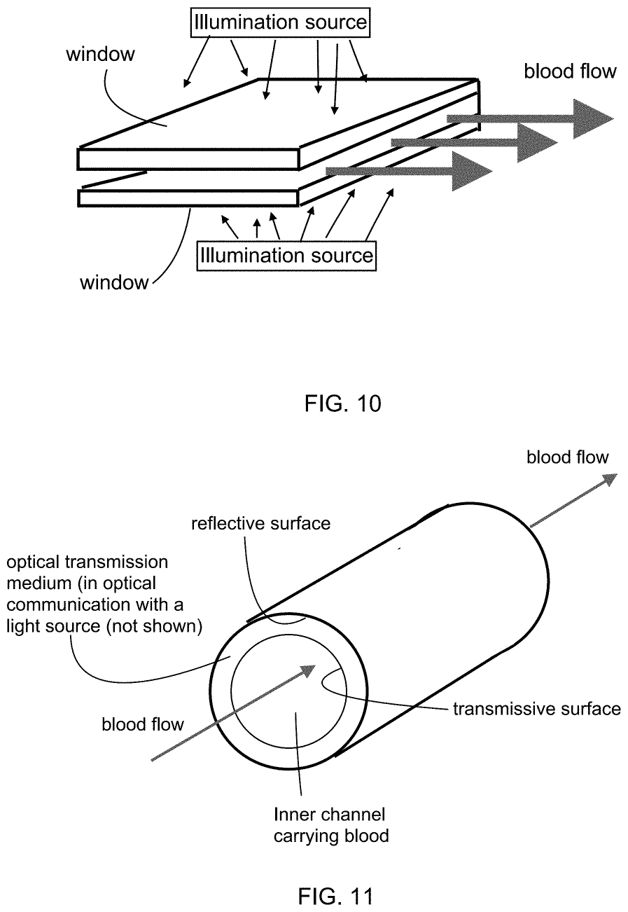

FIG. 10 shows a perspective view of an illumination device. In this embodiment the device has two windows that are transmissive to the illumination light. The windows form a planar channel through which the blood flows. The planner channel increases the surface area and decreases the depth of penetration into the blood that the light must travel.

FIG. 11 shows a perspective view of an illumination device. The device is a hollow fiber optic. The blood flows through the inner channel of the fiber, while the light is transmitted along the length of the fiber in the outer wall of the fiber. The fiber wall has an outer reflective surface and an inner transmissive surface, that directs the light inwardly to the flowing blood. The transmissivity/reflectivity of the inner surface can be adjusted to enable the light to also propagate along the length of the fiber and thus provide illumination, and activation, along the length of the fiber.

In an embodiment the illumination source may be constructed to deliver an illumination light dosage to the blood through the skin, without requiring the removal of the blood form the body. In this embodiment the wavelength of the light, the illumination pattern, and the beam profile of the light may be selected such that the light passes through the skin and circulatory structures, without tissue damage, and then have sufficient energy in the blood to activate the photosensitizer to produce ROS. An example of this embodiment would be a cuff link illumination device for illuminating a patient's wrist area. The light source of these illumination devices may be a coherent light source such as a laser or a non-coherent light source. The light source may have a narrow wavelength, be a broad-spectrum wavelength, an array of diode lasers and other source of light. The light source preferably provides light at a wavelength that is optimized for activation of the photosensitizer, transmission through the blood, and both.

The present inventions further relate to nanocompositions. In particular, the present inventions provide nanocompositions for clinical (e.g., targeted therapeutic), diagnostic (e.g., imaging), and research applications in the field of virology, and generally to relating to pathogens.

An embodiment of the present inventions is a composition having a core molecule, to which a pathogen specific TA (targeting agent) and a PS (photosensitizer) are linked (e.g., chemically, covalently or otherwise attached). In preferred embodiments, the photosensitizer is a phthalocyanine dye, and the core molecule is a multi-arm nanoparticle, a linear molecule, PEG, a multi-arm PEG, 8PEG, 8PEGA, 8PEGMAL, or combinations thereof. These embodiments are used to provide pathogenic PDT.

The targeting agent (TA) can be an agent e.g., peptide, antibody, protein, or small molecule, that targets a pathogen. As such these targeting agents may be referred to as Pathogen specific targeting agents (PSTA). Pathogen targeting peptides (PTP) in embodiments may be a preferred TA. The TA's, are linked to a nanoparticle to form a nanocomposition that also may have a PS. The TA nanoparticle composition may be used for imaging. The TAs are specific to a particular pathogen, or spices, group of family of pathogens. The TA can bind to, target or be specific for unique identifiers, e.g., structures, on the pathogen. The PSTA nanocomposition is transduced into or otherwise affixed to the pathogen at much higher levels than it is transduced into or affixed to other tissues and cells, such as, for example, red blood cells, liver, kidney, lung, skeletal muscle, cardiac, epithelial or brain. In certain embodiments the ratio of selectivity of PSTA nanocomposition for the pathogen relative to all other tissues and cells present in the patient, is at least 2:1 and greater, is at least 3:1 and greater, is at least 4:1 and greater, is at least 10:1 and greater, and is at least 100:1 and greater.

The photoactive agent can be any dye or molecule that produces or causes the production of ROS when exposed to light or produces other compounds when exposed to light that kill, destroy or render inert, the pathogen. Examples of PS include, for example, IR700, methylene blue (MB), chlorin e6 (Ce6), Coomassie blue, gold, or combinations thereof.

An embodiment of the present nanocompositions is a nanoparticle, a phthalocyanine PS, and a PSTA. This embodiment is used to provide pathogenic PDT.

An embodiment of the present nanocompositions is a nanoparticle, a phthalocyanine PS, where the phthalocyanine is a phthalocyanine die disclosed and taught in U.S. Pat. No. 7,005,518, and a PSTA. This embodiment is used to provide pathogenic PDT.

An embodiment of the present nanocompositions is a nanoparticle, a phthalocyanine PS, and a PSTA. This embodiment is used to provide pathogenic PDT.

An embodiment of the present nanocompositions is a nanoparticle, where the nanoparticle is PEG, and preferably 8PEGA, a phthalocyanine PS, and a PSTA. This embodiment is used to provide pathogenic PDT.

An embodiment of the present nanocompositions is a nanoparticle, where the nanoparticle is PEG, and preferably 8PEGA, a phthalocyanine PS, where the phthalocyanine is a phthalocyanine die disclosed and taught in U.S. Pat. No. 7,005,518, and a PSTA. This embodiment is used to provide pathogenic PDT.

An embodiment of the present nanocompositions is a nanoparticle, where the nanoparticle is PEG, and preferably 8PEGA, a phthalocyanine PS, where the phthalocyanine is a phthalocyanine die disclosed and taught in U.S. Pat. No. 7,005,518, and a PSTA. This embodiment is used to provide pathogenic PDT.

As used herein 8PEG refers to and would include any 8-arm polyethylene glycol (PEG) molecule (e.g., nanoparticle). 8PEG would include all 8PEGs where one or more of the end groups of the arms is modified. For example, 8PEG would include 8PEGA (8PEG-A, and similar terms) which is 8PEG having amine terminated end groups on the arms (one, two and preferably all arms). For example, 8PEG would include 8PEGMAL (8PEG-MAL and similar terms) which is 8PEG having maleimide terminated end groups on the arms (one, two and preferably all arms). These 8PEGs would include nanoparticles having a hydrodynamic diameter (e.g., size) of 25 nm and less, a hydrodynamic diameter of 10 nm and less, and having a hydrodynamic diameter of from about 30 nm to about 5 nm and having a hydrodynamic diameter of from about 20 nm to about 5 nm. These 8PEGs would include nanoparticles that are 20 kilodaltons (kDa) and greater, that are 40 kDa and greater, and that are from about 15 kDa to about 50 kDa, and that are from about 5 kDa to about 100 kDa.

IRDye 700DX NHS Ester (“IR700”) is a preferred photosensitizer for the present embodiments of nanocompositions and for the treatment of pathogen conditions using the present embodiments of the targeted nanoparticle and nanocompositions based photodynamic therapies. IR700 is available from LI-Cor and is an embodiment disclosed in U.S. Pat. No. 7,005,518, the entire disclosure of which is incorporated herein by reference. IR700 is a phthalocyanine dye that has minimal sensitive to photobleaching and is thus preferred to many other organic fluorochromes. IR700 has the chemical formula C₇₄H₉₆N₁₂Na₄O₂₇S₆Si₃, has a molecular weight of 1954.21 g/mol, has an exact mass of 1952.37, has a maximum absorbance of light at 689 nm, and also shows much smaller absorbance peaks at 350 nm, and 625 nm. IR700 is also water soluble and salt tolerant. IR700 has the structure of Structure 1:

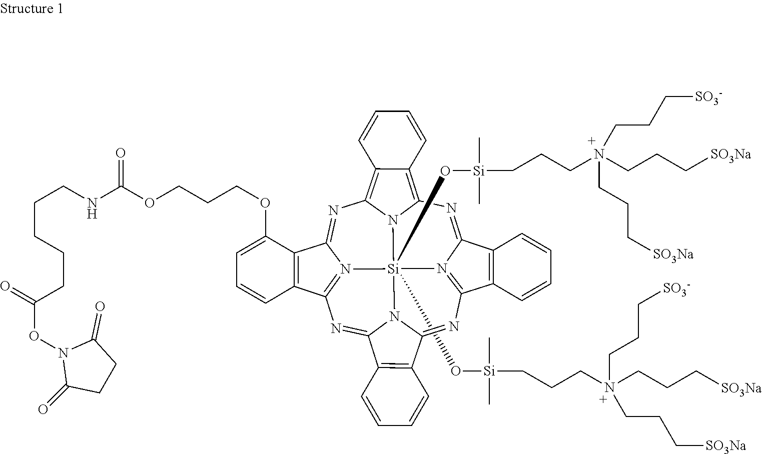

In embodiments the pathogen targeted nanoparticle with IR700 is activated by delivering, to the pathogen tissue having this nanoparticle, light having a wavelength of from about 550 nm to about 750 nm, light having a wavelength of about 300 to 400, light having wavelengths of about 350 nm about 625 nm and about 689 nm, light from about 600 nm to about 800 nm, light from bout 650 nm to about 725 nm, light from about 675 nm to about 725 nm, light at about 689 nm, light at 689 nm, and all wavelength within these ranges, as well as higher and lower wavelengths. In an embodiment the light is provided by a laser and is a laser beam. Preferably, the power of the laser beam, and the amount of energy delivered to the pathogen tissue by the laser beam is below, and well below (e.g., at least 10% below, at least 20% below, at least 50% below) the threshold where the laser beam will heat, damage or cause laser induced optical breakdown. In a preferred embodiment the light that is delivered is eye safe.

Embodiments of the present nana constructs may provide improved methods of treating pathogen conditions. For example, in some embodiments, the present nanocompositions provide a method of treating (e.g., killing) a pathogen, comprising a) contacting an animal with a nanoparticle comprising a matrix, a toxic agent (e.g., photosensitizer), and a pathogen targeting moiety; and b) administering an activator of the toxic agent (e.g., light) to at least a portion of the pathogens in of the animal to activate the toxic agent. In some embodiments, administering the activator kills the pathogen only where activator is administered and only to pathogen or a specific area where the pathogen may be or directed to. In some embodiments, the activator is light. In some embodiments, light from a laser. In some embodiments, the pathogen targeting moiety is a pathogen targeting peptide (PTP). In some embodiments, the photosensitizer is IR700. In some embodiments, the contacting is via intravenous administration. In some embodiments, the pathogen targeting moiety specifically targets viruses. In some embodiments, the nanoparticle is a PEG molecule (e.g., 8-arm PEG). In some embodiments, the nanoparticle is approximately 10 nm or less in size.

In some embodiments, the method further comprises the step of imaging the nanoparticles in the animal. In some embodiments, the imaging is performed after the administering of activator and optionally determines a treatment course of action (e.g., further administering of activator, location of treatment and/or nanoparticles). In further embodiments, the present invention provides compositions and kits comprising the aforementioned nanoparticles and any additional components necessary, sufficient or useful in pathogen ablation and imaging.

In yet other embodiments, the present invention provides the use of the aforementioned nanoparticles (e.g., in pathogen killing). In still further embodiments, the present invention provides systems comprising a) the aforementioned nanoparticles; and b) an instrument for delivery of activator (e.g., a laser or ultrasound instrument). In some embodiments, systems further comprise imaging components (e.g., to image or bound to nanoparticles in pathogens) and computer software and computer processor for controlling the system. In some embodiments, the computer software and computer processor are configured to control the delivery of the activator, image the nanoparticle, and displaying an image of the nanoparticle.

US Patent Publication No. 2015/0328315 teaches and disclose photodynamic therapies, nanocompositions, targeted nanocompositions, imaging and theranostics, the entire disclosure of which is incorporated herein by reference. The photosensitizer (PS) can be any dye or molecule that produces ROS when exposed to light or produces other compounds when exposed to light that kill the pathogen. Examples of photoactive agents include, for example, methylene blue (MB), chlorin e6 (Ce6), Coomassie blue, or gold, and combinations thereof.

The PS can be the compositions disclosed and taught in U.S. Pat. Nos. 8,562,944, 8,906,343, and 9,045,488.

The PS can be PHOTOFRIN,

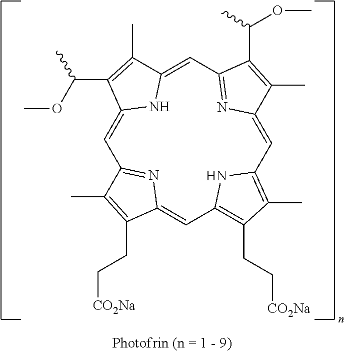

The PS can be Photochlor (CAS #149402-51-7)

The PS can be from of Table 1.

TABLE 1
Photosensitizers

| | | WAVELENGTH, |
|---|---|---|
| PHOTOSENSITIZER | STRUCTURE | nm |
| Porfimer sodium (Photofrin) (BPD) | Porphyrin | 630 |
| ALA | Porphyrin | 635 |
| | precursor | |
| ALA esters | Porphyrin | 635 |
| | precursor | |
| Temoporfin (Foscan) (mTHPC) | Chlorine | 652 |
| Verteporfin | Chlorine | 630 |
| HPPH | Chlorin | 665 |
| SnEt2 (Purlytin) | Chlorin | 660 |
| Talaporfin (LS11, MACE, NPs6) | Chlorin | 660 |
| Ce6-PVP (Fotolon, Ce6 derivitives | Chlorin | 660 |
| (Radachlorin, Photodithazi) | | |
| Silicon phthalocyanine (Pc4) | Phthalocyanine | 675 |
| Padoporfin (TOOKAD) | Bacteriochlorin | 762 |
| Motexafin lutetium (Lutex) | Texaphyrin | 732 |

Embodiments of the present nanocompositions, including 8PEG-CPT nanocompositions, have a PS that is a dye having the following formula of Structure 2:

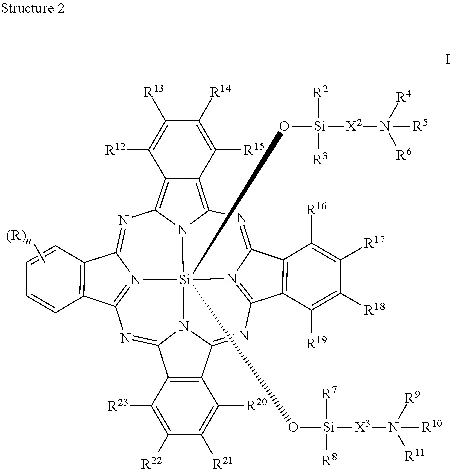

wherein: R is a member selected from the group consisting of -L-Q and -L-Z¹; L is a member selected from the group consisting of a direct link, or a covalent linkage, wherein said covalent linkage is linear or branched, cyclic or heterocyclic, saturated or unsaturated, having 1-60 atoms selected from the group consisting of C, N, P, O, and S, wherein L can have additional hydrogen atoms to fill valences, and wherein said linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds; Q is a reactive or an activatable group; Z¹ is a material; n is 1 or 2; R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkenoyl, optionally substituted alkoxy carbonyl, optionally substituted alkyl carbamoyl, and a chelating ligand, wherein at least one of R⁴, R⁵, R⁶, R⁹, R¹⁰, and R¹¹ comprises a water soluble group; R¹², R¹³, R¹⁴, R¹⁵, R¹⁶R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring; and X² and X³ are each members independently selected from the group consisting of C₁-C₁₀ alkylene optionally interrupted by a heteroatom, wherein if n is 1, the phthalocyanine may be substituted either at the 1 or 2 position and if n is 2, each R may be the same or different, or alternatively, they may join to form a 5- or 6-membered ring.

Embodiments of the present nanocompositions, including 8PEGA-CPT nanocompositions have a PS that is a dye having the following formula of Structure 3:

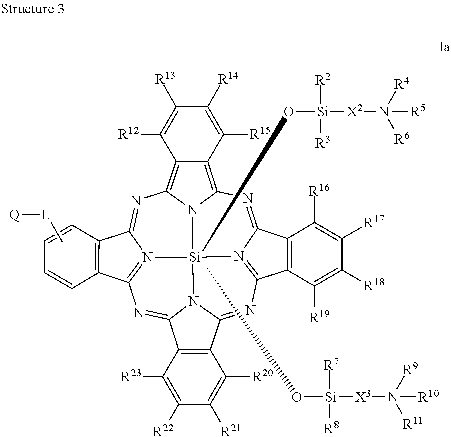

wherein: R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkenoyl, optionally substituted alkoxy carbonyl, optionally substituted alkyl carbamoyl, wherein at least one of R⁴, R⁵, R⁶, R⁹, R¹⁰, and R¹¹ comprises a water soluble group; and R¹², R¹³, R¹⁴, R⁵, R¹⁶R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring.

In embodiments L, from Structure 3 has the following formula: —R¹—Y—X¹—Y¹— wherein R¹ is a bivalent radical or a direct link; Y and Y¹ are each independently selected from the group consisting of a direct link, oxygen, an optionally substituted nitrogen and sulfur; and X¹ is a member selected from the group consisting of a direct link and C₁-C₁₀ alkylene optionally interrupted by a heteroatom. In further embodiments, R¹ is a bivalent radical selected from the group consisting of optionally substituted alkylene, optionally substituted alkyleneoxycarbonyl, optionally substituted alkylenecarbamoyl, optionally substituted alkylenesulfonyl, optionally substituted alkylenesulfonylcarbamoyl, optionally substituted arylene, optionally substituted arylenesulfonyl, optionally substituted aryleneoxycarbonyl, optionally substituted arylenecarbamoyl, optionally substituted arylenesulfonylcarbamoyl, optionally substituted carboxyalkyl, optionally substituted carbamoyl, optionally substituted carbonyl, optionally substituted heteroarylene, optionally substituted heteroaryleneoxycarbonyl, optionally substituted heteroarylenecarbamoyl, optionally substituted heteroarylenesulfonylcarbamoyl, optionally substituted sulfonylcarbamoyl, optionally substituted thiocarbonyl, a optionally substituted sulfonyl, and optionally substituted sulfinyl.

In further embodiments of Structure 3, R¹ is R², R³, R⁷, and R⁸ are each independently selected from optionally substituted alkyl, and optionally substituted aryl, R⁴, R⁵, R⁶, R⁹, R¹⁰, and R¹¹, if present, are each members independently selected from an optionally substituted alkyl, wherein at least two members of the group consisting of R⁴, R⁵, R⁶, R⁷, R⁸, and R⁹ comprise a water soluble functional group; R¹², R¹³, R¹⁴, R¹⁵, R¹⁶R¹⁷, R¹⁸, R¹⁹, R²⁰, R²¹, R²² and R²³ are each hydrogen, halogen, optionally substituted alkylthio, optionally substituted alkylamino and optionally substituted alkoxy, or in an alternative embodiment, at least one of R¹³, R¹⁴, and the carbons to which they are attached, or R¹⁷, R¹⁸, and the carbons to which they are attached, or R², R²² and the carbons to which they are attached, join to form a fused benzene ring; X¹, X² and X³ are each members independently selected from the group consisting of C₁-C₁₀ alkylene optionally interrupted by a heteroatom; and Y and Y¹ are each independently selected from the group consisting of a direct link, oxygen, an optionally substituted nitrogen and sulfur.

In further embodiments of Structure 3, R², R³, R⁷, and R⁸ are each independently selected from optionally substituted methyl, ethyl, and isopropyl; R⁴, R⁵, R⁶, R⁹, R¹⁰, and R¹¹, if present, are each members independently selected from an optionally substituted alkyl, wherein at least two members of the group consisting of R⁴, R⁵, R⁶, R⁷, R⁸, and R⁹ comprise a substituent selected from the group consisting of a carboxylate (—CO₂⁻) group, a sulfonate (—SO₃⁻) group, a sulfonyl (—SO₂⁻) group, a sulfate (—SO₄⁻²) group, a hydroxyl (—OH) group, a phosphate (OPO₃-2) group, a phosphonate (—PO₃⁻²) group, an amine (NH₂) group and an optionally substituted quaternized nitrogen with each having an optional counter ion; R¹², R¹³, R¹⁴, R¹⁵, R¹⁶R¹⁷, R¹⁸, R¹⁹, R²⁰, R²¹, R²² and R²³ are each hydrogen; X¹, X² and X³ are each members independently selected from the group consisting of C₁-C₁₀ alkylene optionally interrupted by a heteroatom; and Y and Y¹ are each independently selected from the group consisting of a direct link, oxygen, an optionally substituted nitrogen and sulfur.

Embodiments of the present nanocompositions, including 8PEGA-CPT nanocompositions have a PS that is a dye having the following formula of Structure 4:

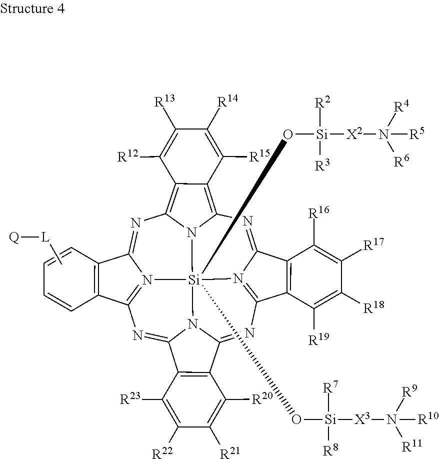

wherein Q is a reactive or an activatable group selected from the group consisting of an alcohol, an activated ester, an acyl halide, an alkyl halide, an optionally substituted amine, an anhydride, a carboxylic acid, a carbodiimide, hydroxyl, iodoacetamide, an isocyanate, an isothiocyanate, a maleimide, an NHS ester, a phosphoramidite, a platinum complex, a sulfonate ester, a thiol, and a thiocyanate.

Embodiments of the present nanocompositions, including 8PEGA-CPT nanocompositions have a PS that is a dye having the following formula of Structure 5:

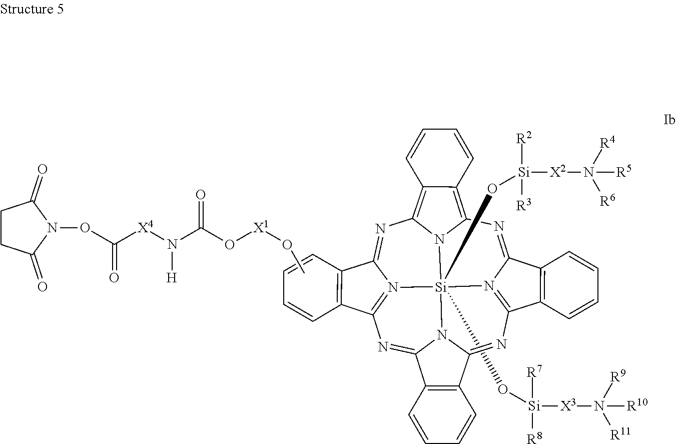

wherein X⁴ is a C₁-C₁₀ alkylene optionally interrupted by a heteroatom.

Embodiments of the present nanocompositions, including 8PEGA-CPT nanocompositions have a PS that is a dye having the formula of Structure 6:

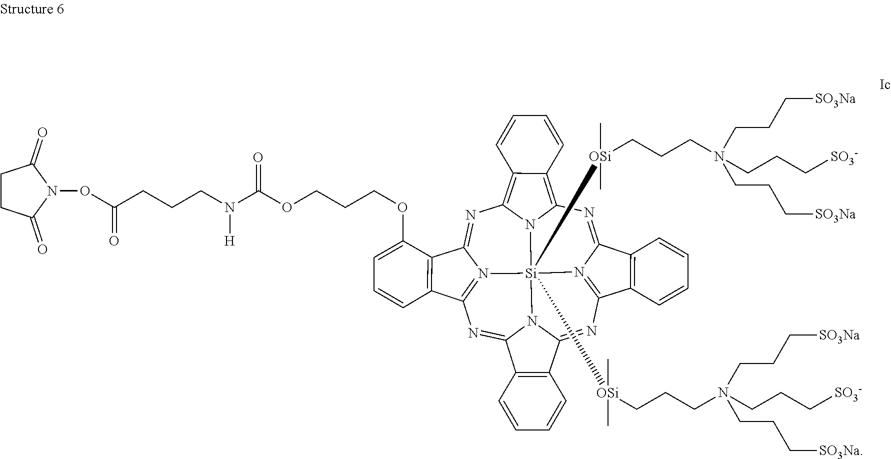

Embodiments of the present nanocompositions, including 8PEGA-CPT nanocompositions have a PS that is a dye having the following formula of Structure 7:

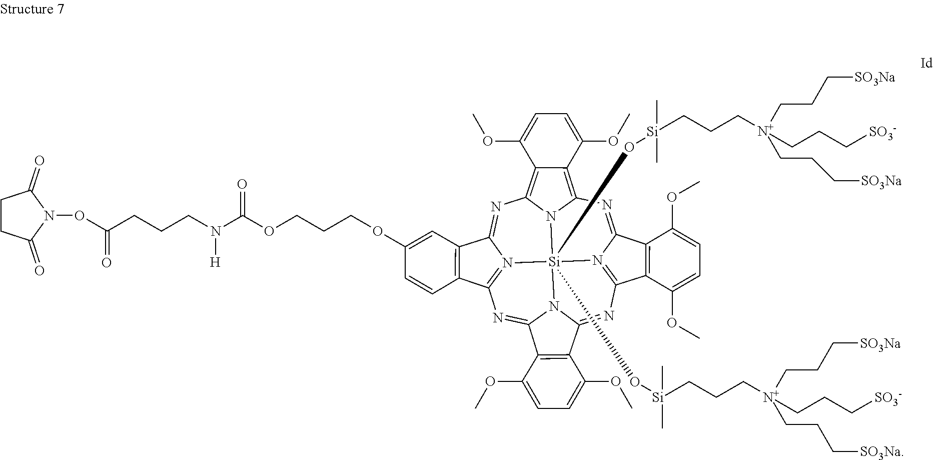

Embodiments of the present nanocompositions, including 8PEGA-CPT nanocompositions have a PS that is a dye having the following formula of Structure 8:

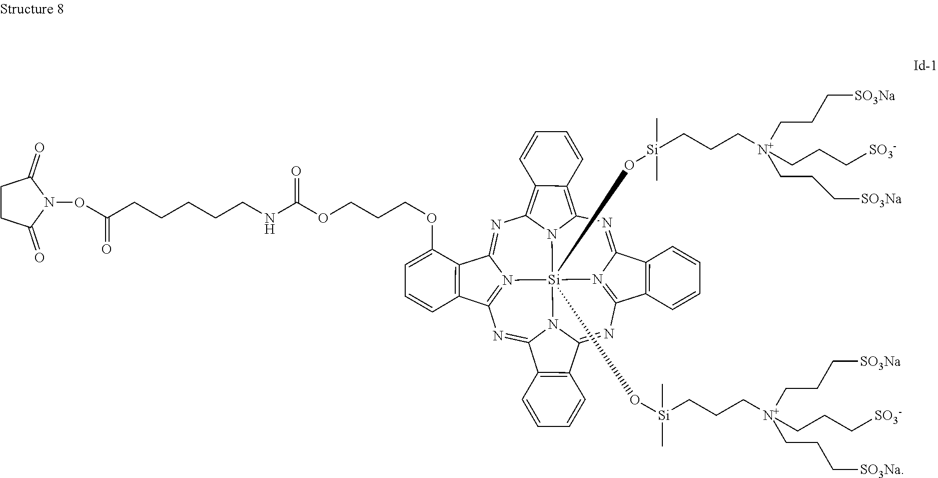

Embodiments of the present nanocompositions, including 8PEGA-CPT nanocompositions have a PS that is a dye having the following formula of Structure 9:

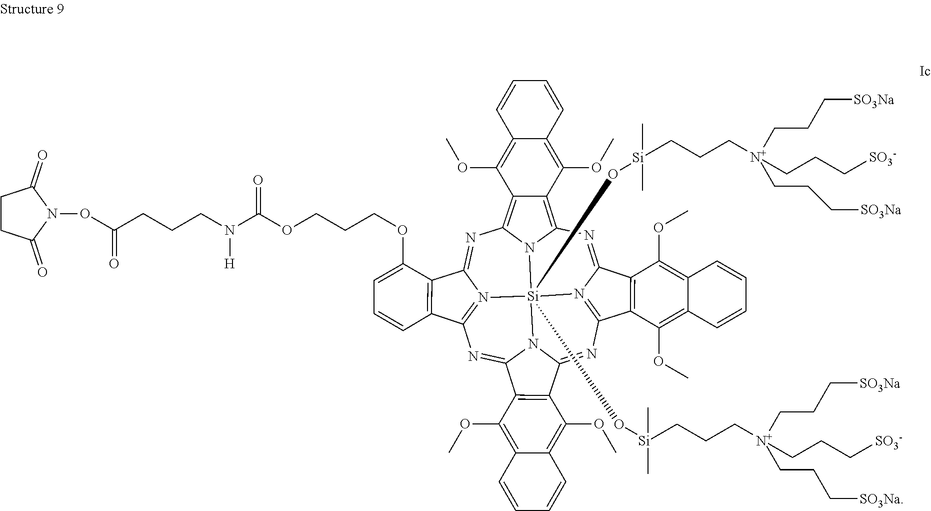

Embodiments of the present nanocompositions, including 8PEGA-CPT nanocompositions have a PS that is a dye having the following formula of Structure 10:

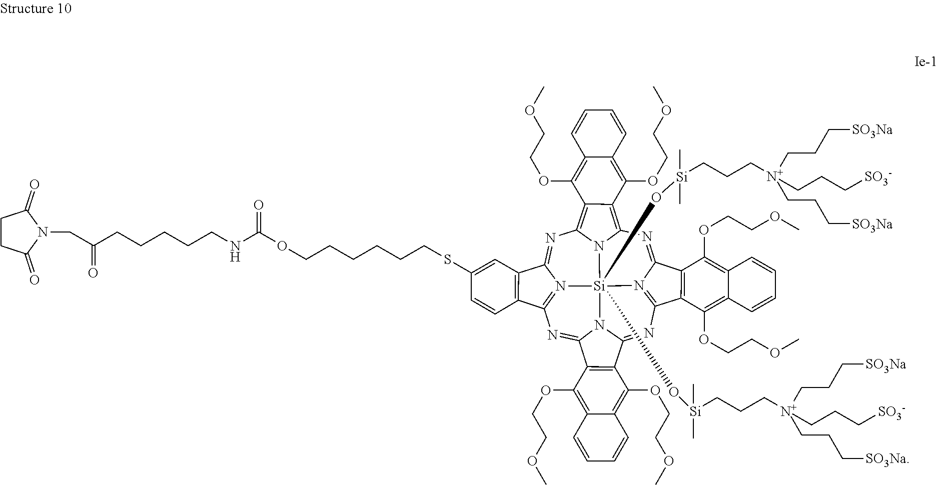

Embodiments of the present nanocompositions, including 8PEGA-CPT nanocompositions have a PS that is a dye having the following formula of Structure 11:

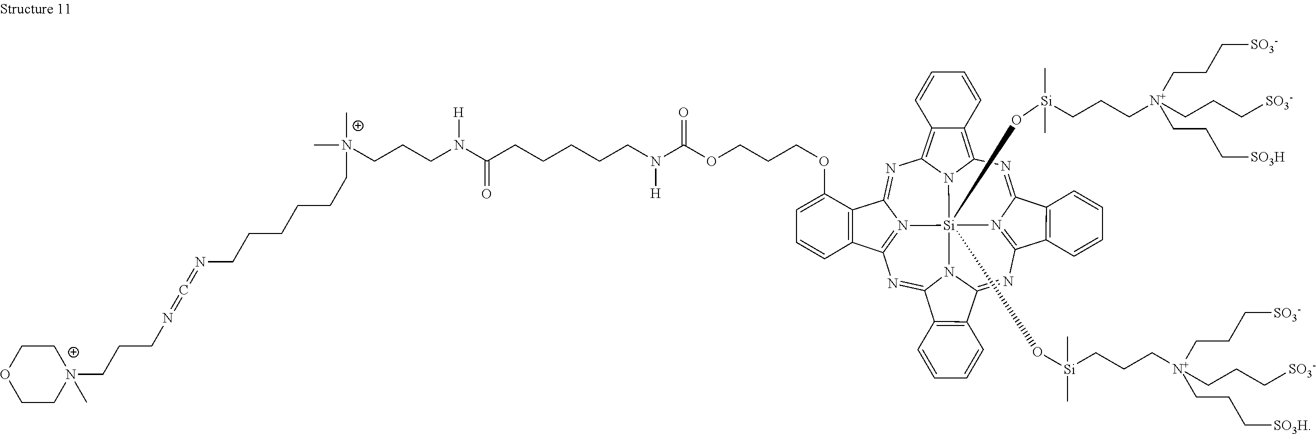

Embodiments of the present nanocompositions, including 8PEGA-CPT nanocompositions have a PS that is a dye having the following formula of Structure 12:

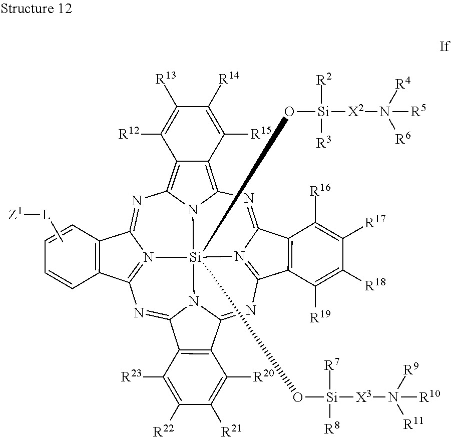

wherein. Z¹ is the nanoparticle; L is a member selected from the group consisting of a direct link, or a covalent linkage, wherein said covalent linkage is linear or branched, cyclic or heterocyclic, saturated or unsaturated, having 1-60 atoms selected from the group consisting of C, N, P, O, and S, wherein L can have additional hydrogen atoms to fill valences, wherein said linkage contains any combination of ether, thiether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds; R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkenoyl, optionally substituted alkoxy carbonyl, optionally substituted alkyl carbamoyl, and a chelating ligand, wherein at least one of R⁴, R⁵, R⁶, R⁹, R¹⁰, and R¹¹ comprises a water soluble group; R¹², R¹³, R¹⁴, R¹⁵, R¹⁶R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring; and X² and X³ are each members independently selected from the group consisting of C₁-C₁ alkylene optionally interrupted by a heteroatom.

In general, and typically, PSs do not have any general affinity for specific tissues, other than certain classes generally favoring rapidly dividing cells (e.g., chlorins in cancer). Thus, in embodiments a targeted delivery of PDT, is beneficial, and in situations necessary to achieve a high contrast ratio between the target tissue, e.g., the tissue to be ablated and bystander tissues, e.g., the tissue that is intended to be unaffected by, and not damaged by, the PDT.

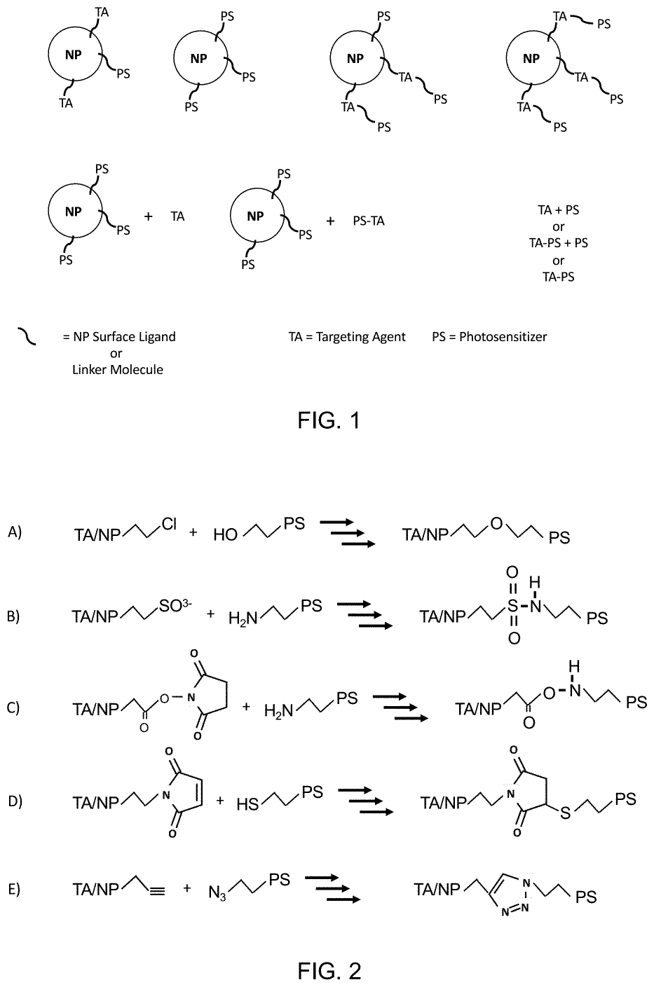

Targeted delivery of a PS may take several different forms: conjugation of a PS to a nanoparticle (NP), conjugation of a PS to a targeting agent (TA), conjugation of both a PS and TA to a NP (the PS being on the NP, the TA, or both), co-administration of a PS (with or without a NP) with a TA, or any combination thereof. Examples of some of these configurations for the present nanocompositions is shown in FIG. 1.

PSTAs include, for example, a small molecule, a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations and variations of these.

Turning to FIG. 2 there is shown embodiments of methods by which a PS may be covalently conjugated to a TA or NP. These methods are useful and applicable across most combinations, and so they are generally discussed as if they are a single method. Thus, any given method of NP conjugation should also be viable for TA conjugation. It further being understood that as a general requirement the functional groups employed should match each other. Tables 2-4 show a list of pairings and the resulting bonds formed between a TA, NP, or PS for examples of embodiments of combinations for embodiments of the present nanocompositions.

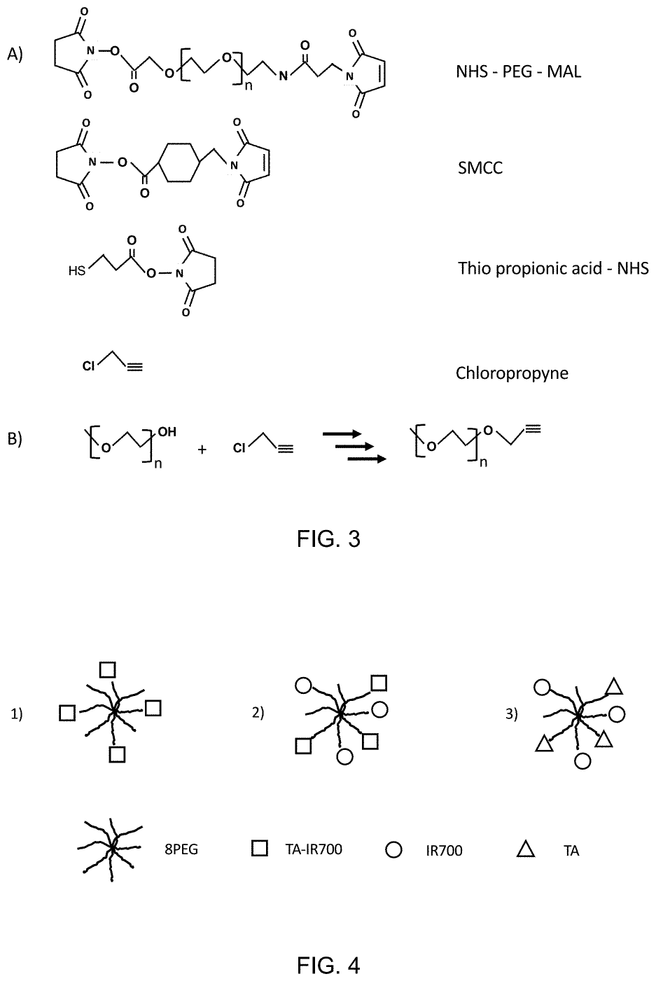

Optionally, conjugation of the PS to a TA, NP, or both, may include a spacer or linker molecule or group. Typically, this will not change the chemistry employed, but it can be used to convert functional groups from one set to another (e.g., an alcohol may be converted to an alkyne with a linking group to enable a different reaction protocol). The linkers may originate on the PS, TA, NP, or any combination, and may be a small molecule chain or polymer. FIG. 3 shows some example linkers and an end group conversion.

An embodiment of a final product would be a NP of small hydrodynamic diameter, preferably from a family of linear, branched, or cyclic macropolymers. Proteins may also be used as they can be small enough, however, they may have competing pharma co-kinetic behavior with the TA. Examples of macropolymers for the NP would include polyethylene glycol (PEG), poly amidoamine (PAMAM), polyethyleneimine (PEI), polyvinyl alcohol, polymethacrylic acid, polymethyl methyl methacrylate (PMMA), polyacrylamide, and poly L-lysine. The preferred platform is PEG, specifically 8-arm branched PEG (8PEG), because of its widely known non-toxicity.

The various embodiments of the nanocompositions disclosed and taught herein can use or have multi-arm PEG NPs, this would include 8PEG and other numbers of arms, including 4-arm PEG, including 4PEGA (amine terminated end groups on the arms (one, two and preferably all arms)) and 4PEGMAL (having maleimide terminated end groups on the arms (one, two and preferably all arms)) and 6-arm PEG (including 6PEGA (amine terminated end groups on the arms (one, two and preferably all arms)) and 6PEGMAL (having maleimide terminated end groups on the arms (one, two and preferably all arms)).

In an embodiment PEG, in particular 8PEG, conjugation can include both a TA and IR700 and may take, for example, the 3 Forms as shown in FIG. 4. FIG. 4, Form 1) has a TA-IR700 conjugate that is attached to 8PEGA to provide a TA-PS-NP nanocomposition, having four IR700-TA conjugates attached to the 8PEGA. FIG. 4, Form 2) is a TA-NP-TA-PS nanocomposition. Form 2) has three TA-IR700 conjugates attached to the 8PEGA and has three IR700 dye molecules attached to the 8PEGA. FIG. 4, Form 3) is a TA-NP-PA nanocomposition. Form 3) has three IR700 dye molecules attached to the 8PEGA and has three TAs attached to the 8PEGA. These forms do not have TAs and PSs bonded to every arm of the 8PEGA. Thus, Form 1) has three unbonded, or open, or non-active arms. Forms 2) and 3) have two unbonded, or open, or non-active arms. The unbonded arms, typically have end or terminus groups that are, for example, cysteine.

Additionally, the order of conjugation of a TA or IR700 to 8PEG is generally interchangeable for Forms 2) and 3); in this manner the IR700s can be attached first and then the TAs, or the TAs first and then the IR700s. A preferred embodiment would be Form 3), with the order of attachment being, attaching IR700s to 8PEG first, and then attaching the TAs to the 8PEGA. A benefit of this preferred method, among others, is to permit all 8PEGs to have at least one IR700 attached without risking the functionality of the TA by further modifying it.

Contrary to the general teaching of the art, it has been discovered that increasing the number of PS attached to the NP does not necessarily increase the amount of ROS produced and does not necessarily increase the efficacy of the nanocomposition. Thus, for situations having four or more PS attached to an NP, and in particular 8PEGA, the ROS production and the efficacy of the nanocomposition may be decreased when compared to a nanocomposition having three or less PS. It is theorized that this occurs because of several facts relating to the spacing of the PS, and thus their ability to produce ROS from the in-situ oxygen.

Thus, embodiments of IR700-8PEGA-PTP nanocompositions have from 1-2 IR700 dyes per 8PEGA, and 3-5 PTPs per 8PGEA. These and other embodiments can have a ratio of PTP to IR700 that is 2.5 to 1 and greater, 3 to 1 and greater, and 5 to 1 and greater. These and other embodiments can have 1, 2, 3, and 4 free arms and more. It being understood that embodiments having lower rations of PTP to IR700 per 8PEGA may also be utilized, including rations of 2 to 1 and 1 to 1. All combinations and variations of these configurations are also contemplated.

Thus, and generally, embodiments of PS-NP-TA nanocompositions have from 1-2 PS per 8PEGA, and 3-5 TA per 8PGEA. Embodiments of these, and other, nanocompositions have a ratio of TA to PS per NP that is 2.5 to 1 and greater, 3 to 1 and greater, and 5 to 1 and greater. These and other embodiments can have 1, 2, 3, and 4 free arms and more. It being understood that embodiments having lower rations of TA to PS per NP may also be utilized, including rations of 2 to 1 and 1 to 1. All combinations and variations of these configurations are also contemplated.

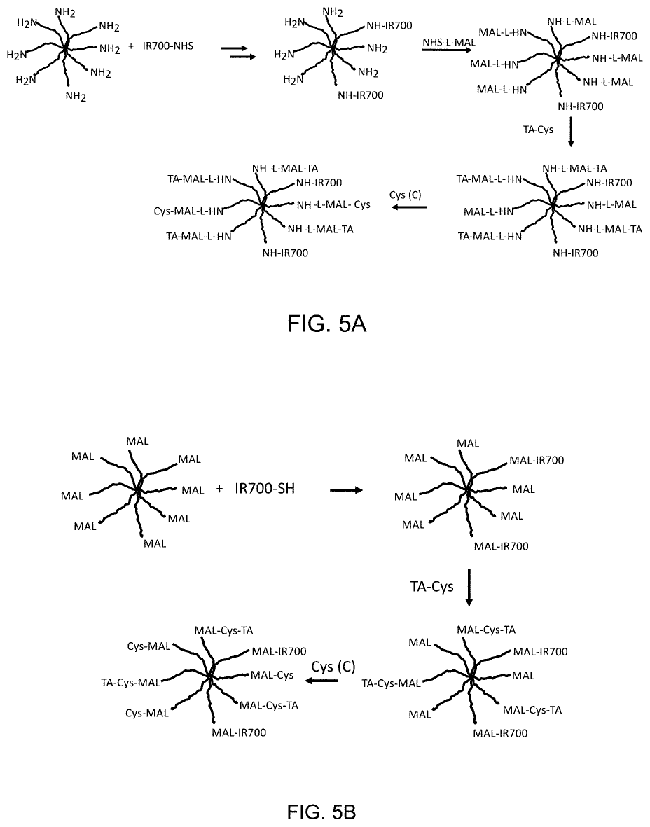

Turning to FIG. 5A there is provided an embodiment of a method to produce the nanocomposition of FIG. 4, Form 3). FIG. 5A illustrates the following steps: IR700-NHS is added to 8PEG-Amine (8PEGA), a linker (L) is added to 8PEGA to convert the amines to maleimides (MAL), IR700-8PEGM is treated with thiol terminated (preferably cysteine, cys) TA, and additional free cysteine is added to cap unreacted MAL groups.

Turning to FIG. 5B there is provided an embodiment of a method to produce the nanocomposition of FIG. 4, Form 3). FIG. 5B has the following steps: IR700-SH is added to 8PEGMAL, IR700-8PEGMAL is treated with thiol terminated TA (preferably cysteine, cys), and additional free cysteine is added to cap unreacted MAL groups.

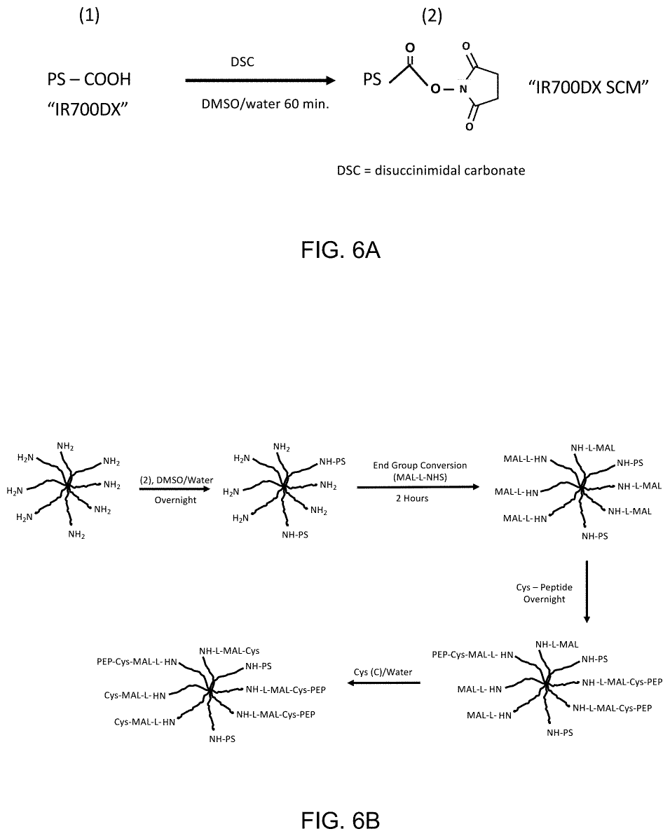

Turning to FIGS. 6A and 6B there is shown a general process for forming targeted nanocompositions for PDT, including an IR700-NP-PTP nanocomposition. “PEP”, (a peptide), is the TA. The end group conversions step of FIG. 6B uses a chemical such as SMCC, BiPEG, or others, that converts the 8PEGA amines to maleimides (“MAL”).

FIG. 6A shows the preparation of the NHS ester (SCM, i.e., succinimidyl ester) for the PS, IR700 (formula (2)). FIG. 6B shows the preparation of the nanocomposition using the HHS ester (FIG. 6A, formula (2)) and a PEP TA.

Covalent conjugation of a NP-X, PS-L-Q, or TA-Z in any combination may take many forms; generally, the entities should have X, Q, and Z functional groups that are reactive towards each other. X, Q, and Z include, but are not limited to alkyl halides, acyl halides, aromatic phenyls, aromatic halides (preferably iodo), carboxylic acids, sulfonic acids, phosphoric acids, alcohols (preferably primary), maleimides, esters, thiols, azides, aldehydes, alkenes (mono or diene), isocyanates, isothiocyanates, amines, anhydrides, or thiols. Tables 2-4 show the matching relevant combinations of NP-X, PS-L-Q, and TA-Z functional groups for conjugation.

TABLE 2
X and Q pairings of NP-X and PS-L-Q for covalent conjugation [Makes PS(L)-NP-X]

| | | | Covalent |
|---|---|---|---|
| NP-X | PS-L-Q | Conditions | Bond |
| Alkyl Halide (Chlorine) | PS-OH | Base, CHCl₃ or DMSO | Ether |
| | PS-SH | | Thio Ether |
| | PS-COOH | | Ester |
| | PS-NH₂ | | |
| Acyl Halide (Chlorine) | PS-NH₂ | 1.5:1 Base:PS-Y (Opt) | Amide |
| | PS-SH | CHCl₃ or DMSO | Thio Ester |
| | PS-OH | | Ester |
| | PS-Phenyl | | Ketone |
| Aromatic (Phenyl) | PS-Cl | AlCl₃, CHCl₃ or DMSO | Alkyl chain |
| | PS-COCl | | ketone |
| Aromatic (Halide Phenyl) | PS-NH₂ | Base, CHCl₃ or DMSO | Secondary Amine |
| | PS-OH | | Ether |
| | PS-SH | | Thioether |
| Carboxylic Acid | PS-OH | Acid, CHCl₃ or DMSO; | Ester |
| | PS-NH₂ | Acid, CHCl₃ or DMSO; | Amide |
| | PS-Cl | Base, CHCl₃ or DMSO; | Ester |
| | PS-SH | Acid, CHCl₃ or DMSO | Thioester |
| Sulfonic Acid | PS-OH | 1.5:1 Base:PS-Y | Sulfonic ester |
| | PS-NH₂ | PCl₅, CHCl₃ or DMSO; | Amino Sulfonate |
| | PS-SH | SOCl₂ may also be used | Sulfonic thioester |
| Phosphoric Acid | PS-OH | 1.5:1 Base:PS-Y | Phosphoramidite |
| | PS-NH₂ | SOCl₂, CHCl₃ or DMSO | |
| | PS-SH | | |
| Alcohol (Primary) | PS-Cl | Base, CHCl₃ or DMSO; | Ether |
| | PS-COOH | Base, CHCl₃ or DMSO; | Ester |
| | PS-ester | Base, CHCl₃ or DMSO; | Ester |
| | PS-thioester | Base, CHCl₃ or DMSO; | Ester |
| | PS-anhydride | Base, CHCl₃ or DMSO; | Ester |
| | PS-CHO | Base, Pd catalyst, CHCl₃; | Ester |
| | PS-ITC | 1.5:1 Base:PS-Y, CHCl₃; | Thiocarbamate |
| | PS-IC | 1.5:1 Base:PS-Y, CHCl₃ | Urethane |
| Maleimide (MAL) | PS-SH | pH 6-8 in water; | Thioether |
| | | 1.5:1 Base:PS-Y inorganic solvent | |
| Ester | PS-NH₂ | Acid, CHCl₃ or DMSO | Amide |
| | PS-OH | | Ester |
| | PS-SH | | Thioester |
| Thiol | PS-Mal | pH 6-8 in water; | Thioether |
| | PS-ITC | 1.5:1 Base:PS-Y, CHCl₃; | Dithiocarbamate |
| | PS-IC | 1.5:1 Base:PS-Y, CHCl₃ | Thiourethane |
| Azide | PS-Alkyne | Cu(I), CHCl₃ or DMSO; | Triazole |
| | | Cu free, CHCl₃ or water | |
| Aldehyde | PS-NH2 | CuI, TBHP, CHCl₃; | Amide |
| | PS-OH | Base, Pd catalyst, CHCl₃; | Ester |
| Alkene | PS-Diene | Diels-Alder | Cyclo-alkyl |
| Alkyne | PS-Azide | Cu(I), CHCl₃ or DMSO; | Triazole |
| | | Cu free, CHCl₃ or water | |
| isocyanate | PS-OH | Base, CHCl₃; | Urethane |
| | PS-NH₂ | CHCl₃; | Urea |
| | PS-SH | Base, CHCl₃ | Thiourethane |
| isothiocyanate | PS-SH | 1.5:1 Base:PS-Y, CHCCl₃; | Dithiocarbamate |
| | PS-NH₂ | pH 7.4 in water; | Thiourea |
| | PS-OH | 1.5:1 Base:PS-Y, CHCl₃ | Thiocarbamate |
| Amine (A) | PS-COOH | Acid, CHCl₃ or DMSO; | Amide |
| | PS-COCl | Base (Opt), CHCl₃ | Amide |
| | PS-NHS | pH 7.4 in water; | Amide |
| | PS-CHO | Base, Pd catalyst, CHCl₃; | Amide |
| | PS-ITC | pH 7.4 in water; | Thiourea |
| | PS-IC | pH 7.4 in water | Urea |
| Anhydride | PS-NH₂ | CHC13 or DMSO; | Amide |
| | PS-OH | 1.5:1 Base:PS-Y, CHCl₃; | Ester |
| | PS-SH | 1.5:1 Base:PS-Y, CHCl₃ | Thioester |
| Thiol | PS-SH | Oxidant, CHCl₃ | Disulfide |
| Aromatic (Phenyl) | TA-Cl | AlCl₃, CHCl₃ or DMSO | Alkyl chain |
| | TA-COCl | | ketone |
| Aromatic (Halide Phenyl) | TA-NH₂ | Base, CHCl₃ or DMSO | Secondary Amine |
| | TA-OH | | Ether |
| | TA-SH | | Thioether |
| Carboxylic Acid | TA-OH | Acid, CHCl₃ or DMSO; | Ester |
| | TA-NH₂ | Acid, CHCl₃ or DMSO; | Amide |
| | TA-Cl | Base, CHCl₃ or DMSO; | Ester |
| | TA-SH | Acid, CHCl₃ or DMSO | Thioester |
| Sulfonic Acid | TA-OH | 1.5:1 Base:PS-Y | Sulfonic ester |
| | TA-NH₂ | PCl₅, CHCl₃ or DMSO; | Amino Sulfonate |
| | TA-SH | SOCl₂ may also be used | Sulfonic thioester |
| Phosphoric Acid | TA-OH | 1.5:1 Base:PS-Y | Phosphoramidite |
| | TA-NH₂ | SOCl₂, CHCl₃ or DMSO | |
| | TA-SH | | |
| Alcohol (Primary) | TA-Cl | Base, CHCl₃ or DMSO; | Ether |
| | TA-COOH | Base, CHCl₃ or DMSO; | Ester |
| | TA-ester | Base, CHCl₃ or DMSO; | Ester |
| | TA-thioester | Base, CHCl₃ or DMSO; | Ester |
| | TA-anhydride | Base, CHCl₃ or DMSO; | Ester |
| | TA-CHO | Base, Pd catalyst, CHCl₃; | Ester |
| | TA-ITC | 1.5:1 Base:PS-Y, CHCl₃; | Thiocarbamate |
| | TA-IC | 1.5:1 Base:PS-Y, CHCl₃ | Urethane |
| Maleimide (Mal) | TA-SH | pH 6-8 in water; | Thioether |
| | | 1.5:1 Base:PS-Y in organic solvent | |
| Ester | TA-NH₂ | Acid, CHCl₃ or DMSO | Amide |
| | TA-OH | | Ester |
| | TA-SH | | Thioester |
| Thiol | TA-Mal | pH 6-8 in water; | Thioether |
| | TA-ITC | 1.5:1 Base:PS-Y, CHCl₃; | Dithiocarbamate |
| | TA-IC | 1.5:1 Base:PS-Y, CHCl₃ | Thiourethane |
| Azide | TA-Alkyne | Cu(I), CHCl₃ or DMSO; | Triazole |
| | | Cu free, CHCl₃ or water | |
| Aldehyde | TA-NH2 | CuI, TBHP, CHCl3; | Amide |
| | TA-OH | Base, Pd catalyst, CHCl₃; | Ester |
| Alkene | TA-Diene | Diels-Alder | Cyclo-alkyl |
| Alkyne | TA-Azide | Cu(I), CHCl₃ or DMSO; | Triazole |
| | | Cu free, CHCl₃ or water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:PS-Y, CHCl₃; | Dithiocarbamate |
| | TA-NH₂ | pH 7.4 in water; | Thiourea |
| | TA-OH | 1.5:1 Base:PS-Y, CHCl₃ | Thiocarbamate |
| Amine (A) | TA-COOH | Acid, CHCl₃ or DMSO; | Amide |
| | TA-COCl | Base (Opt), CHCl₃ | Amide |
| | TA-NHS | pH 7.4 in water; | Amide |
| | TA-CHO | Base, Pd catalyst, CHCl₃; | Amide |
| | TA-ITC | pH 7.4 in water; | Thiourea |
| | TA-IC | pH 7.4 in water | Urea |

TABLE 3
X and Z pairings of PS(L)-NP-X or NP-X alone and
TA-Z for covalent conjugation [to make PS(L)-
NP-TA the preferred material or NP-TA alone]

| PS(L)-NP-X | | | Covalent |
|---|---|---|---|
| (or NP-X) | TA-Z | Conditions | Bond |
| Alkyl Halide | TA-OH | Base, CHCl₃ or DMSO | Ether |
| (Chlorine) | TA-SH | | Thio Ether |
| | TA-COOH | | Ester |
| | TA-NH₂ | | |
| Acyl Halide | TA-NH₂ | 1.5:1 Base:PS-Y (Opt) | Amide |
| (Chlorine) | TA-SH | CHCl₃ or DMSO | Thio Ester |
| | TA-OH | | Ester |
| | TA-Phenyl | | Ketone |
| Anhydride | TA-NH₂ | CHCl3 or DMSO; | Amide |
| | TA-OH | 1.5:1 Base:PS-Y, CHCl₃; | Ester |
| | TA-SH | 1.5:1 Base:PS-Y, CHCl₃ | Thioester |
| Thiol | TA-SH | Oxidant, CHCl₃ | Disulfide |

*Opt = optional; NHS = N-hydroxy succinimide; ITC = isothiocyanate; IC = isocyanate

TABLE 4
Q and Z pairings of PS-L-Q and TA-Z for covalent conjugation [This
makes PS(L)-TA, that could potentially be used (no NP) or could then
be attached to the NP to form a new (and never tried) form PA-TS-NP]

| | | | Covalent |
|---|---|---|---|
| PS-L-Q | TA-Z | Conditions | Bond |
| Alkyl Halide (Chlorine) | TA-OH | Base, CHCl₃ or DMSO | Ether |
| | TA-SH | | Thio Ether |
| | TA-COOH | | Ester |
| | TA-NH₂ | | |
| Acyl Halide (Chlorine) | TA-NH₂ | 1.5:1 Base:PS-Y (Opt) | Amide |
| | TA-SH | CHCl₃ or DMSO | Thio Ester |
| | TA-OH | | Ester |
| | TA-Phenyl | | Ketone |
| Aromatic (Phenyl) | TA-Cl | AlCl₃, CHCl₃ or DMSO | Alkyl chain |
| | TA-COCl | | ketone |
| Aromatic (Halide Phenyl) | TA-NH₂ | Base, CHCl₃ or DMSO | Secondary Amine |
| | TA-OH | | Ether |
| | TA-SH | | Thioether |
| Carboxylic Acid | TA-OH | Acid, CHCl₃ or DMSO; | Ester |
| | TA-NH₂ | Acid, CHCl₃ or DMSO; | Amide |
| | TA-Cl | Base, CHCl₃ or DMSO; | Ester |
| | TA-SH | Acid, CHCl₃ or DMSO | Thioester |
| Sulfonic Acid | TA-OH | 1.5:1 Base:PS-Y | Sulfonic ester |
| | TA-NH₂ | PCl₅, CHCl₃ or DMSO; | Amino Sulfonate |
| | TA-SH | SOCl₂ may also be used | Sulfonic thioester |
| Phosphoric Acid | TA-OH | 1.5:1 Base:PS-Y | Phosphoramidite |
| | TA-NH₂ | SOCl₂, CHCl₃ or DMSO | |
| | TA-SH | | |
| Alcohol (Primary) | TA-Cl | Base, CHCl₃ or DMSO; | Ether |
| | TA-COOH | Base, CHCl₃ or DMSO; | Ester |
| | TA-ester | Base, CHCl₃ or DMSO; | Ester |
| | TA-thioester | Base, CHCl₃ or DMSO; | Ester |
| | TA-anhydride | Base, CHCl₃ or DMSO; | Ester |
| | TA-CHO | Base, Pd catalyst, CHCl₃; | Ester |
| | TA-ITC | 1.5:1 Base:PS-Y, CHCl₃; | Thiocarbamate |
| | TA-IC | 1.5:1 Base:PS-Y, CHCl₃ | Urethane |
| Maleimide (Mal) | TA-SH | pH 6 - 8 in water; | Thioether |
| | | 1.5:1 Base:PS-Y inorganic solvent | |
| Ester | TA-NH₂ | Acid, CHCl₃ or DMSO | Amide |
| | TA-OH | | Ester |
| | TA-SH | | Thioester |
| Thiol | TA-Mal | pH 6-8 in water; | Thioether |
| | TA-ITC | 1.5:1 Base:PS-Y, CHCl₃; | Dithiocarbamate |
| | TA-IC | 1.5:1 Base:PS-Y, CHCl₃ | Thiourethane |
| Azide | TA-Alkyne | Cu(I), CHCl₃ or DMSO; | Triazole |
| | | Cu free, CHCl₃ or water | |
| Aldehyde | TA-NH2 | CuI, TBHP, CHCl3; | Amide |
| | TA-OH | Base, Pd catalyst, CHCl₃; | Ester |
| Alkene | TA-Diene | Diels-Alder | Cyclo-alkyl |
| Alkyne | TA-Azide | Cu(I), CHCl₃ or DMSO; | Triazole |
| | | Cu free, CHCl₃ or water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:PS-Y, CHCl₃; | Dithiocarbamate |
| | TA-NH₂ | pH 7.4 in water; | Thiourea |
| | TA-OH | 1.5:1 Base:PS-Y, CHCl₃ | Thiocarbamate |
| Amine (A) | TA-COOH | Acid, CHCl₃ or DMSO; | Amide |
| | TA-COCl | Base (Opt), CHCl₃ | Amide |
| | TA-NHS | pH 7.4 in water; | Amide |
| | TA-CHO | Base, Pd catalyst, CHCl₃; | Amide |
| | TA-ITC | pH 7.4 in water; | Thiourea |
| | TA-IC | pH 7.4 in water | Urea |
| Anhydride | TA-NH₂ | CHCl3 or DMSO; | Amide |
| | TA-OH | 1.5:1 Base:PS-Y, CHCl₃; | Ester |
| | TA-SH | 1.5:1 Base:PS-Y, CHCl₃ | Thioester |
| Thiol | TA-SH | Oxidant, CHCl₃ | Disulfide |

*Opt = optional; NHS = N-hydroxy succinimide; ITC = isothiocyanate; IC = isocyanate

The present inventions relate generally to targeted photodynamic therapies, targeted photosensitizers, including targeted nano constructs and uses of these therapies and materials in dynamic therapies for treating, managing, reducing and eliminating pathogens in body fluids and from animals including humans. In particular, in an embodiment, the present inventions related to the removal of pathogens from circulating blood in animals, including humans. The processes and systems disclosed herein may include any of the various features disclosed herein, including one or more of the following embodiments.

Statement 1: A nanocomposition comprising: a. a photosensitizer (PS), wherein the photosensitizer is a phthalocyanine dye; b. a nanoparticle (NP); wherein the nanoparticle is 8PEG; and, a targeting agent (TA), wherein the targeting agent is a pathogen targeting peptide (PTP).

Statement 2: The nanocomposition of statement 1, wherein the nanocomposition is configured to provide a photodynamic therapy for a pathogen indication.

Statement 3: A nanocomposition, for use in treating a pathogen condition, the nanocomposition comprising: a. a photosensitizer (PS), wherein the photosensitizer is a phthalocyanine dye; b. a nanoparticle (NP); wherein the nanoparticle is selected from the group of 8PEG, 8PEGA and 8PEGMAL; and c. a targeting agent (TA), wherein the targeting agent is a pathogen targeting peptide (PTP); d. wherein the nanocomposition is configured for providing a photodynamic therapy for the pathogen condition.

Statement 4: The nanocomposition of statement 3, wherein the nanocomposition has less than 3 PS per NP.

Statement 5: A method of treating a pathogen condition, using the nanocomposition of any of statements 1 to 4, the method comprising: administering to an animal a plurality of any of the nanocompositions of statements 1 to 4; waiting a sufficient time for the nanocompositions to accumulate in a targeted pathogen tissue of the animal; and, illuminating the targeted pathogen tissue with light having a wavelength and sufficient energy to activate the PS, thereby producing reactive oxygen species (ROS).

Statement 6: The method of statement 5, wherein the light is a laser beam.

Statement 7: The method of statement 5, wherein the illumination of the targeted pathogen tissue results in less than a 5 degree C. raise in temperature of the illuminated tissue.

Statement: 8: The method of statement 5, wherein the illumination of the targeted pathogen tissue results in less than a 2 degree C. raise in temperature of the illuminated tissue.

Statement 9: The method of statement 5, wherein the illumination of the targeted pathogen tissue does not raise the temperature of the illuminated tissue.

Statement 10: The method of statement 5, wherein the illumination of the targeted pathogen tissue does not result in thermal breakdown of the illuminated tissue.

Statement 11: The method of statements 5 to 10, wherein the illumination of the targeted pathogen tissue does not result in induced optical breakdown.

Statement 12: A kit comprising a container having a plurality of the nanocompositions of any of statements 1 to 4 and an illumination light source having a wavelength and power selected to activate the PS.

Statement 13: The kit of statement 12, wherein the illumination light comprises a disposable optical delivery device.

Statement 14: A composition for use in treating a pathogen condition using a photodynamic therapy, the composition comprising: a photosensitizer (PS), wherein the photosensitizer is a phthalocyanine dye; a core molecule; and, a targeting agent (TA), wherein the TA is specific to pathogen tissue.

Statement 15: The composition of statement 14, wherein the composition is a nanocomposition and the core molecule is a nanoparticle NP.

Statement 16: The compositions of any of statements 14-15, wherein the core molecule is selected from the group consisting of PEG, 8PEG, 8PEGA and 8PEGMAL.

Statement 17: The compositions of any of statements 14 to 16, wherein the PS is water soluble.

Statement 18: The compositions of any of statements 14 to 17, wherein the PS, TA and both are directly attached to the core molecule.

Statement 19: The composition of claim 14, wherein the direct attachment is a covalent bond.

Statement 20: The compositions of any of statements 14 to 19 wherein the PS, TA and both are attached to the core by a linking moiety.

Statement 21: The compositions of any of statements 14 to 20, wherein the TA is attached to the core by a linking moiety.

Statement 22: The compositions of any of statements 14 to 21, wherein the TA is attached to the PS.

Statement 23: The compositions of any of statements 14 to 22, wherein the TA is attached to the PS; and wherein the TA is not directly attached to the core.

Statement 24: The compositions of any of statements 14 to 23, wherein the TA and PS form a conjugate, wherein the conjugate is attached to the core.

Statement 25: The compositions of any of statements 14 to 24, wherein the core is an 8PEG nanoparticle, and the 8PEG nanoparticle has one free arm.

Statement 26: The compositions of any of statements 14 to 25, wherein the core is an 8PEG nanoparticle, and the 8PEG nanoparticle has at least two free arms.

Statement 27: The compositions of any of statements 14 to 26, wherein the core is an 8PEG nanoparticle, and the 8PEG nanoparticle has at least three free arms.

Statement 28: The compositions of any of statements 14 to 27, wherein the core is an 8PEG nanoparticle, comprising no more than three PS.

Statement 29: The compositions of any of statements 14 to 28, wherein the core is an 8PEG nanoparticle, comprising no more than two PS.

Statement 30: The compositions of any of statements 14 to 29, wherein the core is an 8PEG nanoparticle, and a ratio of TA to PS is selected from the group consisting of and wherein the 2.5 to 1, 3 to 1, 4 to 1 and 5 to 1.

Statement 31: The compositions of any of the statements 14 to 30, wherein the core is an 8PEG nanoparticle, and wherein the composition has a hydrodynamic diameter selected from the group consisting of 70 nm and less, 50 nm and less, 25 nm and less, and 10 nm and less.

Statement 32: The compositions of any of the statements 14 to 31, wherein the core is an 8PEG nanoparticle, and wherein the nanoparticle has a mass selected from the group consisting of about 10 kDa and greater, about 20 kDa and greater, about 40 kDa and greater, and about 50 kDa and greater.

Statement 33: A method of treating a pathogen condition comprising: administering to an animal a targeted nanoparticle comprising IR700; wherein the nanoparticle comprises a pathogen targeting agent; delivering light in the wavelength range of from about 600 nm to about 800 nm to a pathogen tissue having the target nanoparticle; whereby the IR700 is activated, and the pathogen tissue is destroyed.

Statement 34: The methods of statement 33, wherein the animal is a mammal.

Statement 0.35: The method of statement 34, wherein the animal is a human.

Statement 36: The methods of statements 33-35, wherein the nanoparticle is 8PEGA.

Statement 37: The methods of statements 33-36 wherein the targeting agent is a pathogen specific protein.

Statement 38: The methods of statements 33-37 wherein the targeting agent is a pathogen targeting peptide.

Statement 39: A method of treating a pathogen condition using IR700 comprising: Administering a targeted nanocomposition to a patient, the nanocomposition comprising IR700, a PTP and an 8PEG nanoparticle, whereby the nanocomposition accumulated in a pathogent tissue of the patient.

Statement 40: the method of statement 39 further comprising administering a product comprising IR700 to a patient, whereby the IR700 is delivered to pathogen tissue, and found in only pathogen tissue; and administering light to activate the IR700, thereby producing an ROS.

Statement 41: a method of treating pathogen tissue, comprising: contacting an animal with a nanoparticle comprising a matrix, an active agent, and a pathogen targeting moiety; and administering an activator of said active agent to at least a portion of the pathogen tissue of said animal; wherein the active agent comprises a phthalocyanine dye comprising a luminescent fluorophore moiety having at least one silicon containing aqueous-solubilizing moiety, wherein said phthalocyanine dye has a core atom selected from the group consisting of Si, Ge, Sn, and Al; wherein said phthalocyanine dye exists as a single core isomer, essentially free of other isomers; and has a reactive or activatable group.

Statement 42: A method of treating pathogen tissue, comprising: contacting an animal with a nanoparticle comprising a matrix, an active agent, and a pathogen targeting moiety; and administering an activator of said active agent to at least a portion of the pathogen tissue of said animal; wherein the active agent consists essentially of a phthalocyanine dye comprising a luminescent fluorophore moiety having at least one silicon containing aqueous-solubilizing moiety, wherein said phthalocyanine dye has a core atom selected from the group consisting of Si, Ge, Sn, and Al; wherein said phthalocyanine dye exists as a single core isomer, essentially free of other isomers; and has a reactive or activatable group.

Statement 43: A method of treating pathogen tissue, comprising: contacting an animal with a nanoparticle comprising a matrix, an active agent, and a pathogen targeting moiety; and administering an activator of said active agent to at least a portion of the pathogen tissue of said animal; wherein the active agent consists of a phthalocyanine dye comprising a luminescent fluorophore moiety having at least one silicon containing aqueous-solubilizing moiety, wherein said phthalocyanine dye has a core atom selected from the group consisting of Si, Ge, Sn, and Al; wherein said phthalocyanine dye exists as a single core isomer, essentially free of other isomers; and has a reactive or activatable group.

Statement 44: The methods of statements 40-43, wherein the matrix comprises PEG, and wherein the said core atom is Si.

Statement 45: The methods of statements 40-44, wherein the matrix comprises PEG and wherein said dye has the following formula:

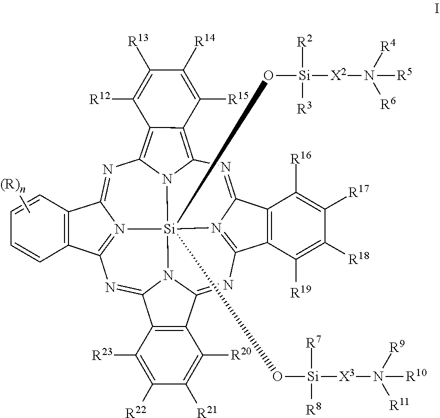

wherein: R is a member selected from the group consisting of -L-Q and -L-Z¹; L is a member selected from the group consisting of a direct link, or a covalent linkage, wherein said covalent linkage is linear or branched, cyclic or heterocyclic, saturated or unsaturated, having 1-60 atoms selected from the group consisting of C, N, P, O, and S, wherein L can have additional hydrogen atoms to fill valences, and wherein said linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds; Q is a reactive or an activatable group; Z¹ is a material; n is 1 or 2; R², R³, R⁷, and R⁸ are each independently selected from optionally substituted alkyl, and optionally substituted aryl; R⁴, R⁵, R⁶, R⁹, R¹⁰, and R¹¹, if present, are each members independently selected from the group consisting of hydrogen, optionally substituted alkyl, optionally substituted alkenoyl, optionally substituted alkoxy carbonyl, optionally substituted alkyl carbamoyl, and a chelating ligand, wherein at least one of R⁴, R⁵, R⁶, R⁹, R¹⁰, and R¹¹ comprises a water soluble group; R¹², R¹³, R¹⁴, R¹⁵, R¹⁶R¹⁷, R¹⁸, R¹⁹, R²⁰, R²¹, R²² and R²³ are each members independently selected from the group consisting of hydrogen, halogen, optionally substituted alkylthio, optionally substituted alkylamino and optionally substituted alkoxy, or in an alternative embodiment, at least one of i) R¹³, R¹⁴, and the carbons to which they are attached, or ii) R¹⁷, R¹⁸, and the carbons to which they are attached, or iii) R²¹, R²² and the carbons to which they are attached, join to form a fused benzene ring; and X² and X³ are each members independently selected from the group consisting of C₁-C₁₀ alkylene optionally interrupted by a heteroatom, wherein if n is 1, the phthalocyanine may be substituted either at the 1 or 2 position and if n is 2, each R may be the same or different, or alternatively, they may join to form a 5- or 6-membered ring.

Statement 46: The methods of statements 40-45, wherein the patient is a human.

Statement 47: The methods of statements 40-45, wherein the animal is a mammal.

### EXAMPLES

The following examples are provided to illustrate various embodiments of systems, processes, compositions, applications and materials of the present inventions. These examples are for illustrative purposes, may be prophetic, and should not be viewed as, and do not otherwise limit the scope of the present inventions.

### Example 1

### Example of Product (PS(L)-NP-TA=IR700-8PEGA-Peptide)

[A=Amine; MAL=maleimide; NHS=N-hydroxy succinimide].

The present invention utilizes the macropolymer 8-arm polyethylene glycol (8PEG-X), a TA (TA-Z), and a PS-L-Q, in any combination. The PS-L-Q is IR700-L-Q and its derivatives, the targeted tissue is a pathogen, and TA is a peptide. In the present specific case, the pathogen is COVID-19, and the corresponding TA is a fragment of ACE2 (ACE2-F, IEEQAKTFLDKFNHEAEDLFYQS).

In one embodiment, TA-Z is conjugated directly with PS-L-Q, where PS-L-Q is IR700-NHS or IR700-MAL. IR700-NHS can be conjugated to the N-terminus of TA-Z or one of the lysine groups directly. IR700-MAL can be conjugated directly to TA-Z that has an added thiol group at the C or N-terminus (e.g. via an additional cysteine), or a lysine group that has been modified to be thiol terminated (e.g. cysteine). The product is a PS-TA conjugation.

In another embodiment, PS-TA-Z is covalently conjugated to 8PEG-X via a thiol-maleimide reaction, preferably X=MAL and Z=thiol; 8PEG-X may begin as a maleimide or start as an amine that is converted to a MAL. Preferably, TA-Z=TA-cys, a cysteine terminated peptide. The product is PS-TA-8PEG.

Optionally, 8PEG-X may be conjugated with IR700-L-Q independently, and then further modified with IR700-TA. The product is PS-TA-8PEG-PS.

In the ideal embodiment, PS-L-Q is IR700-NHS or IR700-SH and 8PEG-X is A or MAL termination. IR700-NHS/SH is conjugated to 8PEG-X, yielding the form of 8PEGA-IR700 or 8PEGMAL-IR700 in a mol ratio that is less than 3:1 IR700:8PEG, but more than 1:1. IR700-8PEG-X is then conjugated to TA-Z, where preferably Z=thiol of cysteine.

ACE2-F and IR700-L-Q may be covalently conjugated with or without 8PEG-X in any combination, including, but not limited to: ACE2-F and IR700 conjugated as separate entities per arm; IR700 conjugated ACE2-F on 8PEG; and IR700 conjugated ACE2-F on IR700 conjugated 8PEG. The preferred combination is to first conjugate IR700-L-Q to 8PEG-X and then attach the TA via 8PEG-X to ensure that at least 1 PS per 8PEG is present and that TA functionality is preserved by minimizing its modification.

### Example of Product (PS(L)-NP-TA=IR700-8PEGA-Peptide

[A=Amine; MAL=maleimide; NHS=N-hydroxy succinimide]

The present invention utilizes the macropolymer 8-arm polyethylene glycol (8PEG-X), a TA (TA-Z), and a PS-L-Q, in any combination. The PS-L-Q is IR700-L-Q and its derivatives, the targeted tissue is a pathogen, and TA is a peptide. In the present specific case, the pathogen is COVID-19, and the corresponding TA is a fragment of ACE2 (ACE2-F, IEEQAKTFLDKFNHEAEDLFYQS).

In one embodiment, TA-Z is conjugated directly with PS-L-Q, where PS-L-Q is IR700-NHS or IR700-MAL. IR700-NHS can be conjugated to the N-terminus of TA-Z or one of the lysine groups directly. IR700-MAL can be conjugated directly to TA-Z that has an added thiol.

group at the C or N-terminus (e.g., via an additional cysteine), or a lysine group that has been modified to be thiol terminated (e.g., cysteine). The product is a PS-TA conjugation.

In another embodiment, PS-TA-Z is covalently conjugated to 8PEG-X via a thiol-maleimide reaction, preferably X=MAL and Z=thiol; 8PEG-X may begin as a maleimide or start as an amine that is converted to a MAL. Preferably, TA-Z=TA-cys, a cysteine terminated peptide. The product is PS-TA-8PEG.

Optionally, 8PEG-X may be conjugated with IR700-L-Q independently, and then further modified with IR700-TA. The product is PS-TA-8PEG-PS.

In the ideal embodiment, PS-L-Q is IR700-NHS or IR700-SH, and 8PEG-X is A or MAL termination. IR700-NHS/SH is conjugated to 8PEG-X, yielding the form of 8PEGA-IR700 or 8PEGMAL-IR700 in a mol ratio that is less than 3:1 IR700:8PEG, but more than 1:1. IR700-8PEG-X is then conjugated to TA-Z, where preferably Z=thiol of cysteine.

ACE2-F and IR700-L-Q may be covalently conjugated with or without 8PEG-X in any combination, including, but not limited to: ACE2-F and IR700 conjugated as separate entities per arm; IR700 conjugated ACE2-F on 8PEG; and IR700 conjugated ACE2-F on IR700 conjugated 8PEG. In an embodiment the combination is to first conjugate IR700-L-Q to 8PEG-X and then

attach the TA via 8PEG-X to ensure that at least 1 PS per 8PEG is present and that TA functionality is preserved by minimizing its modification.

### Example 2

As for EXAMPLE 1 wherein the pathogen is E. coli and the TA is GRHIFWRR.

### Example 3

As for EXAMPLE 1 wherein the pathogen is the Hepatitis B virus, and the TA is LRNIRLRNIRLRNIR.

### Example 4

As for EXAMPLE 1 wherein the pathogen is the Hepatitis B virus, and the TA is LRNIRLRNIRLRNIR.

### Example 5

As for EXAMPLE 1 wherein the pathogen is the Hepatitis C virus, and the TA is MARHRNWPLVMV.

### Example 6

As for EXAMPLE 1 wherein the pathogen is the s. aureus, and the TA is VPHNPGLISLQG.

### Example 7

As for EXAMPLE 1 wherein the pathogen is the west nile virus and the TA is CDVIALLACHLNT.

### Example 8

A treatment protocol using any of the compositions of compositions of the present Examples is as follows: I/V dosing of the patient with the composition (prior to treatment), inserting a “shunt” to circulate blood out of the body, through a device for treatment, and then retuning the blood to the body. The device would “illuminate” the blood with the correct wavelength and power of light to cause the photodynamic destruction of the virus. This simple approach safely removes the possibility of collateral damage to other blood components, destroys circulating virus and potentially promotes a lasting beneficial immune response to the virus.

Additional factor to the illumination device: Take the blood from the arm or other body part, circulate “pump” it through a heated (37° C.) device that has a window (likely all around the tubing) that will illuminate at the right wavelength for PDT and return the blood to the body. The appropriate wavelength to usefully penetrate the blood (650-800 nm) under the conditions of the blood flowing through the device. The size of the illumination chamber is dimensioned to provide a useful power of light based on configuration of the chamber and the flow rate of the blood. The illumination duration x the illumination equals, exceeds the threshold for PDT, while remains below the threshold where blood damage occurs. This time is dependent in part on the flow front and the length of the window.

### Example 9

Using the nanostructure in a therapy to reduce viral load (e.g., COVID-19 viral load) including the steps of dosing of less than or equal to 450 mg/kg particle in humans, and a therapeutic dosage of light administered that does not exceed 85% of the power that would yield thermal breakdown.

### Example 10

Compositions and methods of killing pathogens. In particular examples, the method includes contacting a pathogen having a surface protein with a therapeutically effective amount of an antibody-IR700 molecule, wherein the antibody specifically binds to the surface protein of the pathogen. The blood is removed from the animal and subsequently illuminated, such as at a wavelength of 660 to 740 nm at a dose of for example at least 1 J cm⁻², which activates the IR-700 forming the ROS. The blood after illumination and ROS formation is then returned to the animal. In this process of illumination ROS formation can be done on a continuous basis, such as by using a device similar to a dialysis machine, but with an illumination section that the blood passes through.

For the sake of brevity, only certain ranges are explicitly disclosed herein. However, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present embodiments are well adapted to attain the ends and advantages mentioned as well as those that are inherent therein. The particular embodiments disclosed above are illustrative only, as the present embodiments may be modified and practiced in different but equivalent manners apparent to those skilled in the art having the benefit of the teachings herein. Although individual embodiments are discussed, all combinations of each embodiment are contemplated and covered by the disclosure. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure.

What is claimed:

1. A nanocomposition, for use in treating a pathogen condition, the nanocomposition comprising:
a photosensitizer (PS) comprising a chlorin, a bacteriochlorin, or a phthalocyanine;
a nanoparticle (NP) comprising 8PEG, 8PEGA, or 8PEGMAL; and
a targeting agent (TA), wherein the targeting agent is a pathogen targeting peptide (PTP);
wherein the nanocomposition is configured for providing a photodynamic therapy for the pathogen condition.

2. The nanocomposition of claim 1, wherein the PS is IR700.

3. The nanocomposition of claim 1, wherein the nanocomposition has less than 3 PS per NP.

4. The nanocomposition of claim 1, wherein nanoparticle comprises an 8PEG nanoparticle, and the 8PEG nanoparticle has at least one free arm.

5. The nanocomposition of claim 1, wherein the PS and the TA and are directly bonded to the nanoparticle.

6. The nanocomposition of claim 1, wherein the PS and the TA are covalently bonded to the nanoparticle.

7. The nanocomposition of claim 1, wherein the PS and TA are both are attached to the nanoparticle by a linking moiety.

8. The nanocomposition of claim 1, wherein the nanocomposition has a hydrodynamic diameter selected from the group consisting of 70 nm and less, 50 nm and less, 25 nm and less, and 10 nm and less.

9. The nanocomposition of claim 1, wherein the nanoparticle has a mass selected from the group consisting of about 10 kDa and greater, about 20 kDa and greater, about 40 kDa and greater, and about 50 kDa and greater.

10. A method of treating a pathogen condition comprising:
administering to an animal a nanocomposition comprising:
a photosensitizer (PS) comprising a chlorin, a bacteriochlorin, or a phthalocyanine;
a nanoparticle (NP) comprising 8PEG, 8PEGA, or 8PEGMAL; and
a targeting agent (TA), wherein the targeting agent is a pathogen targeting peptide (PTP); wherein the nanocomposition is configured for providing a photodynamic therapy for the pathogen condition.
waiting a sufficient time for the nanocompositions to accumulate in a targeted pathogen tissue of the animal; and,
illuminating the targeted pathogen tissue with light having a wavelength and sufficient energy to activate the PS, thereby producing reactive oxygen species (ROS) adjacent to the pathogen.

11. The method of claim 10, wherein the light is a laser beam.

12. The method of claim 10, wherein the reactive oxygen species kill the pathogen forming pathogen fragments that stimulate an immune response against the pathogen.

13. The method of claim 10, wherein lasting immunity is provided against the pathogen.

14. The method of claim 10, wherein the pathogen is a virus, bacteria, fungi or parasite.

15. The method of claim 10 wherein the virus is selected from a group containing influenza viruses, corona viruses, SARS-CoV-2, Ebola, HIV, SARS, MERS.

16. The method of claim 10 wherein the bacteria is selected from a group containing gram-positive and gram-negative bacteria.

17. The method of claim 10, wherein the animal is a human.

18. A method of reducing the pathogen load in a patient comprising:
administering a photodynamic therapy (PDT) composition to the patient; wherein the PDT composition comprises a photoactive agent and a targeting agent that specifically targets the pathogen;
binding the PDT composition to the pathogen in the blood;
removing the patients' blood from the patient and illuminating the removed blood with light to activate the photoactive agent, thereby producing produce reactive oxygen species adjacent to the pathogen;
placing the illuminated blood into the patient, whereby the load for the pathogen in the patient is reduced.

19. The method of claim 18, wherein the reactive oxygen species kill the pathogen forming pathogen fragments that stimulate an immune response for the pathogen.

20. The method of claim 18, wherein lasting immunity is provided for the pathogen.

21. A kit comprising:
a container having a plurality of the nanocomposition comprising:
a photosensitizer (PS) comprising a chlorin, a bacteriochlorin, or a phthalocyanine;
a nanoparticle (NP) comprising 8PEG, 8PEGA, or 8PEGMAL; and
a targeting agent (TA), wherein the targeting agent is a pathogen targeting peptide (PTP); wherein the nanocomposition is configured for providing a photodynamic therapy for the pathogen condition; and
an illumination light source having a wavelength and power selected to activate the PS.

22. The kit of claim 21, wherein the illumination light comprises a disposable optical delivery device.
